(19) United States
(12) Patent Application Publication
(10) Pub. No.: US 2023/0404920 A1
(43) Pub. Date: Dec. 21, 2023

(54) MICROFLUIDIC PREPARATION OF FLUOROCARBON NANODROPLETS

(71) Applicants: Bracco Suisse SA, Cadempino (CH); AVIGNON UNIVERSITÉ, Avignon CEDEX 1 (FR)

(72) Inventors: Romain MELICH, Choisy (FR); Philippe BUSSAT, Pers-Jussy (FR); Samir CHERKAOUI, Feigeres (FR); Christiane CONTINO-PEPIN, Althen des Paluds (FR); Stéphane DESGRANGES, Avignon (FR)

(21) Appl. No.: 18/036,018

(22) PCT Filed: Nov. 11, 2021
(86) PCT No.: PCT/EP2021/081432

§ 371 Date: May 9, 2023

(30) Foreign Application Priority Data
Nov. 11, 2020 (EP) 20207039.7

(51) Int. Cl. A61K 9/107; A61K 47/22; A61K 47/18; A61K 47/26; A61K 49/22; A61K 31/02; A61K 9/00

(52) U.S. Cl. CPC A61K 9/1075; A61K 47/22; A61K 47/18; A61K 47/26; A61K 49/226; A61K 31/02; A61K 9/0009

(57) ABSTRACT

The present invention relates to calibrated (per)fluorocarbon nanodroplets comprising an outer layer and an inner core, said outer layer comprising a biocompatible fluorinated surfactant and said inner core comprising a (per)fluorocarbon. The invention further relates to a method of preparation of said calibrated (per)fluorocarbon nanodroplets through microfluidic technique, and to their use for in vivo or in vitro diagnostic and/or for therapy.

### TECHNICAL FIELD

The invention generally relates to calibrated (per)fluorocarbon nanodroplets stabilized by biocompatible fluorinated surfactants and their method of preparation through microfluidic technique. The invention further relates to the use of such calibrated (per)fluorocarbon nanodroplets for in vitro or in vivo diagnostic and/or for therapy.

### BACKGROUND OF THE INVENTION

Phase-change contrast agents (PCCAs) or acoustically activated nanodroplets are receiving increased popularity in both ultrasound diagnostic and therapeutic delivery. Except for the core, often consisting of liquid perfluorocarbons, nanodroplets display similar composition to commercially available gas-filled microbubbles. Owing to Acoustic Droplet Vaporization (ADV) process, encapsulated droplets are converted into gas bubbles upon exposure to ultrasound energy beyond a vaporization threshold. In fact, ultrasounds act as a remote trigger to promote the vaporization of the droplets in a controllable, non-invasive and localized manner. Thanks to their smaller size compared to conventional microbubbles, nanodroplets display prolonged in vivo circulation and deep penetration into the tissues via the extravascular space. Moreover, below vaporization threshold, they are ultrasonically stable with low acoustic attenuation and can be acoustically vaporized at the location of interest.

Perfluorocarbon nanodroplets (“PFC-NDs”) present a real potential as an extravascular ultrasound contrast agent in numerous diagnostic and therapeutic applications including sonopermeabilization, blood brain barrier (BBB) disruption, multimodal imaging modalities and to allow passive (due to the enhanced permeability and retention (EPR) effect in the tumor tissues) or active targeting (by incorporating targeted ligands) for localized delivery of therapeutic drugs or genes. Another potentially valuable characteristic of PFC-NDs is their possible application for novel imaging strategies such as UltraSound Super-Resolution Imaging since these agents can be activated and deactivated on demand by applying intermittent acoustic pulses.

A major limitation of nanodroplets is their relatively limited physico-chemical stability over time, which may affect their use in diagnostic and therapy applications.

A possible strategy to overcome this issue has been identified in the selection of suitable emulsifier.

Most perfluorocarbon droplets produced for imaging purposes are prepared as an emulsion using lipids, surfactants, proteins or diblock polymers as emulsifier (Astafyeva et al, 2015).

Recently, perfluorocarbon nanodroplets stabilized by the biocompatible fluorinated surfactant called “FTAC” have been reported by Astafyeva et al, 2015, who investigated perfluorocarbon emulsions as theranostic agents. In this work, ultrasonic homogenizer was used to produce the perfluorocarbon nanodroplets emulsions.

In the last years, a novel class of biocompatible branched surfactants called “DendriTAC” has been additionally proposed.

WO2016185425 teaches the synthesis and the use of DendriTAC as stabilizers in the preparation of perfluorocarbon nanoemulsions. Standard preparation methods, such as vortex, sonicator and microfluidizer (high pressure homogenizer) are proposed for the emulsion preparation.

Both size and size distribution of nanodroplets are important factors in determining the vaporization threshold, which corresponds to the value of ultrasound pressure required to convert a liquid core droplet into a gaseous bubble. In a polydisperse suspension, characterized by particles of varied sizes, nanodroplets with larger sizes, which require less energy to vaporize than smaller ones, influence the vaporization of the nanodroplets suspension.

On the contrary, in case of a monodisperse system containing particles of relatively uniform size, having a similar and uniform acoustic response to the ultrasound exposure, it is possible to apply the lowest acoustic pressure to achieve the highest vaporization efficiency.

Conventional preparation procedures are routinely applied for the formulation and manufacturing of nanodroplets, including sonication, extrusion, homogenization and microbubble condensation (Sheeran et al. 2017).

More recently, microfluidics (MF) technology, also known as “lab on-a-chip”, has evolved as a powerful and scalable alternative for the consistent preparation of a large variety of size-controlled nanomedicines.

Melich et al, 2020 reports the use of rapid and controlled microfluidic mixing for the manufacturing of PFC-NDs.Up to now, according to Applicants' knowledge, such perfluorocarbon emulsions stabilized by biocompatible fluorinated surfactants have not been prepared yet through microfluidic techniques.

The Applicants have now developed a novel composition comprising calibrated (per)fluorocarbon nanodroplets, stabilized by biocompatible fluorinated surfactants, obtained through microfluidic technique.

Generally, in the state of the art, the term “calibrated” is also indicated as “size-controlled”, “uniform-sized droplets”, “monodisperse(d)” or “monosize(d)”.

Furthermore, the Applicants observed that the molar ratio between fluorinated surfactant molecules (ND shell) and (per)fluorocarbon molecules (ND core) may affect the properties of the calibrated (per)fluorocarbon nanodroplets, in particular of those manufactured according to the microfluidics techniques.

The inventors have in fact surprisingly found that improved stability properties of the NDs can be obtained when using higher molar ratios between said biocompatible fluorinated surfactant and said (per)fluorocarbon, as compared to generally lower molar ratios used in conventional preparations.

### SUMMARY OF THE INVENTION

An aspect of the invention relates to a nanodroplet comprising an outer layer and an inner core, said outer layer comprising a biocompatible fluorinated surfactant and said inner core comprising a fluorocarbon, characterized in that the molar ratio between said biocompatible fluorinated surfactant and said fluorocarbon is higher than 0.06, wherein said biocompatible fluorinated surfactant is selected from

(A) an amphiphilic dendrimer (Dendri-TAC) of generation n comprising:
a hydrophobic central core of valence 2 or 3;
generation chains [symbol] attached to each respective open end of the central core and branching around the core; and
a hydrophilic terminal group at the end of each generation chain;
wherein
n is an integer from 0 to 12 and identifies the hydrophilic terminal group comprising:
a mono-, oligo- or polysaccharide residue,
a cyclodextrin residue,
a peptide residue,
a tris(hydroxymethyl)aminomethane (Tris), or
a 2-amino-2-methylpropane-1,3-diol;
the hydrophobic central core is a group of formula (Ia) or (Ib):

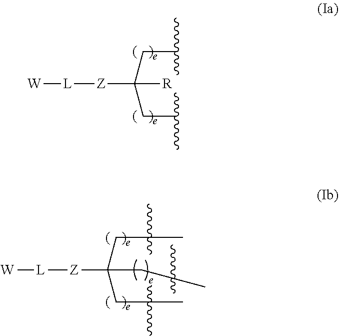

wherein:
W is R_{F} or a group selected from W₀, W₁, W₂ or W₃:

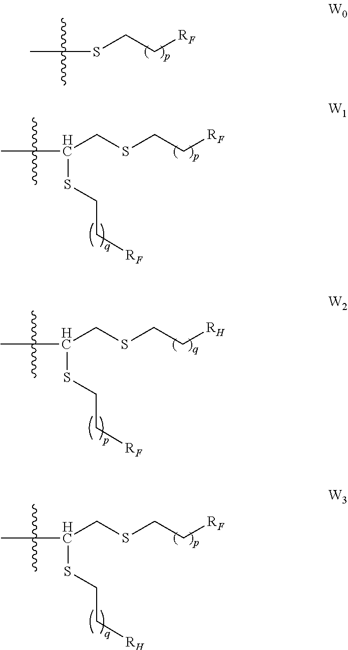

R_{F} is a C₄-C₁₀ perfluoroalkyl,
R_{H} is a C₁-C₂₄ alkyl group,
p is 0, 1, 2, 3 or 4;
q is 0, 1, 2, 3 or 4;
L is a linear or branched C₁-C₁₂ alkylene group, optionally interrupted by one or more —O—, —S—,
Z is C(═O)NH or NHC(═O),
R is a C₁-C₆ alkyl group, and
e is at each occurrence independently selected from 0, 1, 2, 3 or 4,

(B) an amphiphilic linear oligomer (F-TAC) of formula II

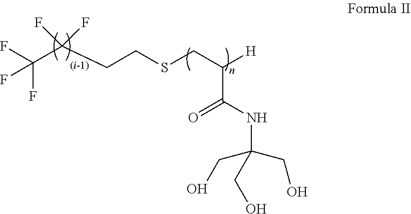

wherein:
   n is the number of repeating Tris units (n=DPn is the average degree of polymerization), wherein the term Tris indicates the tris(hydroxymethyl)aminomethane unit, and
   i is the number of carbon atoms in the fluoroalkyl chain.
or a mixture thereof.
In an embodiment, said fluorocarbon is a perfluorocarbon.

A further aspect relates to an aqueous suspension comprising said nanodroplet.

A preferred embodiment relates to an aqueous suspension comprising a plurality of nanodroplets as above defined, wherein said nanodroplets have a polydispersity index (PDI) lower than 0.25, preferably lower than 0.20, more preferably lower than 0.15, even more preferably lower than 0.10, and a Z-average diameter comprised between 100 nm and 1000 nm, preferably between 120 and 600 nm, more preferably between 150 and 400 nm.

A still further aspect relates to a method for the preparation of an aqueous suspension as defined above, said method comprising the steps of:
a) Preparing an aqueous phase;
b) Preparing an organic phase,

wherein
i) said aqueous phase comprises a biocompatible fluorinated surfactant selected from Dendri-TAC, F-TAC or a mixture thereof and the organic phase comprises a fluorocarbon or
ii) said organic phase comprises a biocompatible fluorinated selected from Dendri-TAC, F-TAC or a mixture thereof surfactant and a fluorocarbon;
c) Injecting said aqueous phase into a first inlet and said organic phase into a second inlet of a microfluidic cartridge, thereby mixing said aqueous phase and said organic phase in a mixing device of the microfluidic apparatus to obtain an aqueous suspension of calibrated fluorocarbon nanodroplets, and
d) Collecting the aqueous suspension of calibrated fluorocarbon nanodroplets from the exit channel of the microfluidic apparatus.

According to a preferred embodiment, said aqueous phase comprises a biocompatible fluorinated surfactant selected from Dendri-TAC, F-TAC or a mixture thereof and said organic phase comprises a fluorocarbon.

Optionally after step d) the collected aqueous suspension is diluted.

A further aspect of the invention is related to a method for the preparation of an aqueous suspension of calibrated fluorocarbon nanodroplets, said method comprising the steps of:
a) Preparing an aqueous phase comprising a biocompatible fluorinated surfactant;
b) Preparing an organic phase comprising a fluorocarbon;
c) Injecting said aqueous phase into a first inlet and said organic phase into a second inlet of a microfluidic cartridge, thereby mixing said aqueous phase and said organic phase in a mixing device of the microfluidic cartridge to obtain an aqueous suspension of calibrated fluorocarbon nanodroplets, and
d) Collecting the aqueous suspension of calibrated fluorocarbon nanodroplets from the exit channel of the microfluidic cartridge.

A still further aspect relates to an aqueous suspension according to the invention for use in a diagnostic and/or therapeutic treatment.

### FIGURES

FIG. 1 is a schematic representation of the core portion of a microfluidic cartridge.

FIG. 2 shows a schematic representation of a cross-section of a staggered herringbone mixer (SHM) design.

### DETAILED DESCRIPTION OF THE INVENTION

The present invention relates to a novel composition comprising calibrated (per)fluorocarbon nanodroplets stabilized by biocompatible fluorinated surfactants, preferably obtained through microfluidic technique. Said calibrated nanodroplets are suitable as contrast agents in ultrasound imaging techniques, known as Contrast-Enhanced Ultrasound (CEUS) Imaging, or in therapeutic applications, e.g. thermal ablation or for ultrasound mediated drug delivery.

An aspect of the present invention relates to a nanodroplet comprising an outer layer and an inner core, said outer layer comprising a biocompatible fluorinated surfactant and said inner core comprising a fluorocarbon, characterized in that the molar ratio between said biocompatible fluorinated surfactant and said fluorocarbon is higher than 0.06.

Biocompatible Fluorinated Surfactants

In the present description and claims, the term “biocompatible” indicates a compound and/or a composition having substantial compatibility with living tissue or a living system by not being toxic, injurious, or physiologically reactive and typically not causing immunological rejection.

In the present description and claims, the expression “surfactant” has its conventional meaning in the chemical field and refers to a compound suitable for forming the stabilizing layer of the nanodroplet.

The expression “fluorinated surfactant” refers to an amphiphilic organic compound suitable for forming the stabilizing layer of the nanodroplets, comprising a hydrophilic moiety and a hydrophobic moiety, said hydrophobic moiety comprising fluorine atoms (i.e. a fluorocarbon part).

The nanodroplets of the present invention are preferably dispersed in an aqueous solvent and stabilized by a layer which is composed of biocompatible fluorinated surfactants advantageously exhibiting a high affinity for both the inner core and surrounding water.

In the present description and claims, the term Dendri-TAC refers to an amphiphilic dendrimer of generation n comprising:
a hydrophobic central core of valence 2 or 3;
generation chains [symbol] attached to each respective open end of the central core and branching around the core; and
a hydrophilic terminal group at the end of each generation chain;
wherein
n is an integer from 0 to 12 and identifies the hydrophilic terminal group comprising:
a mono-, oligo- or polysaccharide residue,
a cyclodextrin residue,
a peptide residue,
a tris(hydroxymethyl)aminomethane (Tris), or
a 2-amino-2-methylpropane-1,3-diol;
the hydrophobic central core is a group of formula (Ia) or (Ib):

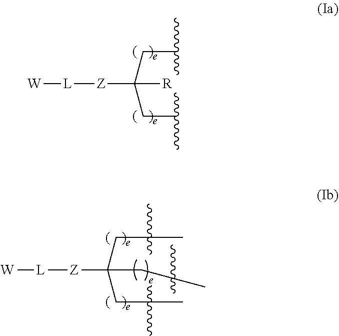

wherein:
W is R_{F} or a group selected from W₀, W₁, W₂ or W₃:

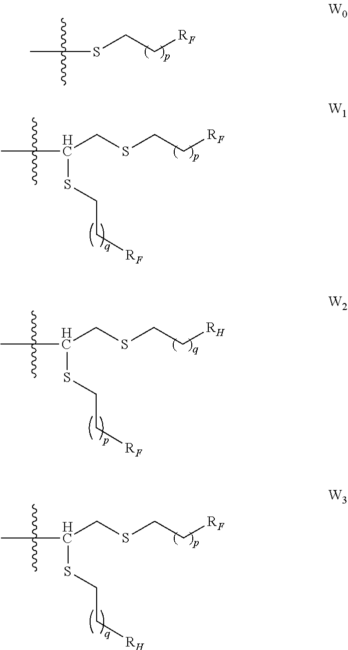

R_{F} is a C₄-C₁₀ perfluoroalkyl,
R_{H} is a C₁-C₂₄ alkyl group,
p is 0, 1, 2, 3 or 4;
q is 0, 1, 2, 3 or 4;
L is a linear or branched C₁-C₁₂ alkylene group, optionally interrupted by
one or more —O—, —S—,
Z is C(═O)NH or NHC(═O),
R is a C₁-C₆ alkyl group, and
e is at each occurrence independently selected from 0, 1, 2, 3 or 4.

In one embodiment, R_{F} is a C₄-C₁₀ perfluoroalkyl and R_{H} is a C₁-C₂₄ alkyl group. In this case, the hydrophobic central core of the amphiphilic dendrimer does comprise a perfluoroalkyl group, and said dendrimer is herein referred to as fluorinated amphiphilic dendrimer.

As used herein, the “valence m of the central core” refers to the number of generation chains attached to the central core, as illustrated in the following scheme 1:

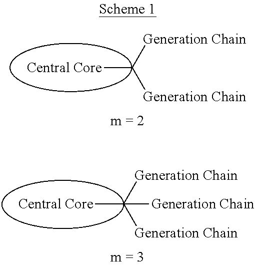

As used herein, a dendrimer of generation n=0, means that the m generation chains are connected to the central core through a first branching point (G₀), corresponding to the valence of the central core. A dendrimer of generation n=1 means that each of the m generation chains ramifies itself once, more specifically at the branching point G₁ (see scheme 2).

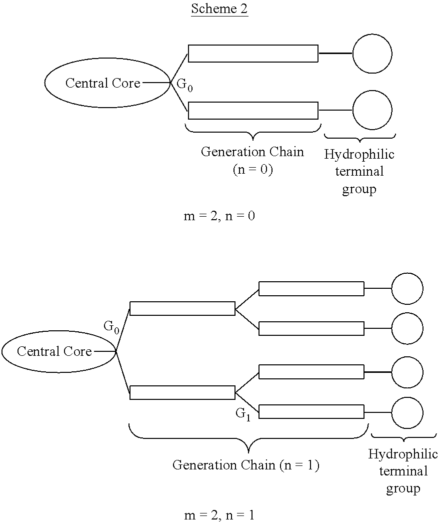

According to preferred embodiments, n is 0, 1 or 2, more preferably n is 0.

Each generation chain of the amphiphilic dendrimers according to the invention is ended by a hydrophilic terminal group.

In this respect, the mono-, oligo- or polysaccharide residue may be notably glucose, galactose, mannose, arabinose, ribose, maltose, lactose, hyaluronic acid.

The cyclodextrin residue may be selected from α, β or γ-Cyclodextrin.

The peptide residue may be chosen from linear or cyclic peptides containing the arginine-glycine-aspartic acid (RGD) sequence.

In another embodiment, there are included dendrimers wherein the generation chains are attached to the central core:
either via the group (a):

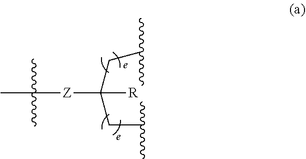

or via the group (b):

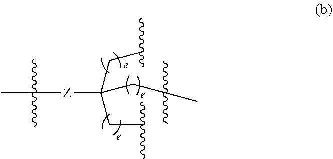

wherein
Z is C(═O)NH or NHC(═O), and is attached to the central core,
R is a C₁-C₆ alkyl group, and
e is at each occurrence independently selected from 0, 1, 2, 3 or 4.

In a further embodiment, there are included dendrimers wherein the central core is a group of formula (Ia) or (Ib):

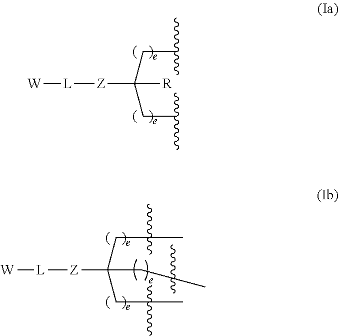

wherein:
W is R_{F} or a group selected from W₀, W₁, W₂ or W₃:

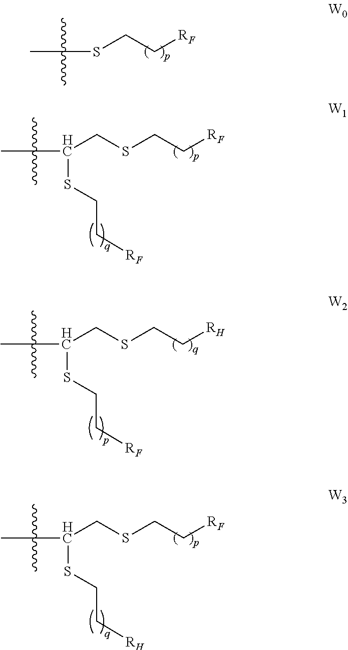

R_{F} is a C₄-C₁₀ perfluoroalkyl,
R_{H} is a C₁-C₂₄ alkyl group,
p is 0, 1, 2, 3 or 4;
q is 0, 1, 2, 3 or 4;
L is a linear or branched C₁-C₁₂ alkylene group, optionally interrupted by one or more —O—, —S—.

In still a further embodiment, there are included dendrimers wherein WL is a group selected from:

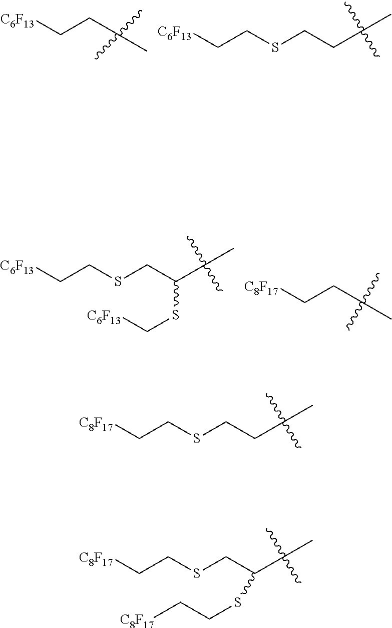

In yet another embodiment, there are included dendrimers wherein each generation chain (n) branches n times via a group (a) or a group (b) as defined above.

In another embodiment, there are included dendrimers wherein the terminal group comprises the following hydrophilic moieties:

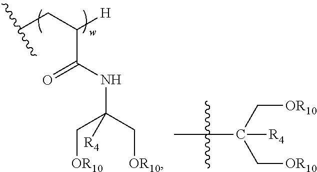

In a particular embodiment, there are included dendrimers having the following formula:

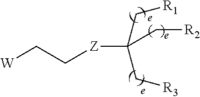

wherein:
   W is R_{F} or a group selected from:

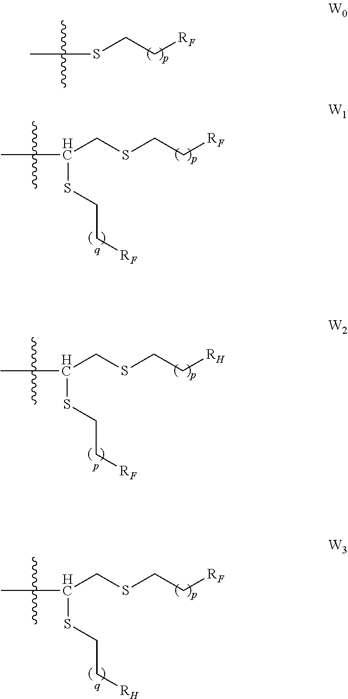

R_{F} being a C₄-C₁₀ perfluoroalkyl and R_{H} being a C₁-C₂₄ alkyl group,
p is 0, 1, 2, 3 or 4;
q is 0, 1, 2, 3 or 4;
Z is (CO)NH or NH(CO);
R₁, R₂, R₃ are H, or a group selected from (c) or (d):

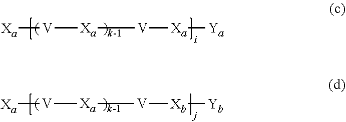

provided that:
R₁, R₂, R₃ are the same and selected from either group (c) or (d) or:
one of R₁, R₂, R₃ is H, the two others being the same and selected from either group (c) or (d);
X is Xₐ when j is 1 and X_{b} when j is 0;
Xₐ is at each occurrence independently selected from —OC(═O) CH₂—NH—, —OC(═O)CH₂—O—CH₂—, —O(CH₂)ᵣC(═O)—NH—, —O(CH₂)ᵣC(═O)—O—CH₂, OC(═O)NH—, —C(═O)—, —NH—, and —OCH₂—;
Yₐ is

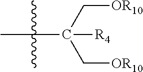

X_{b} is

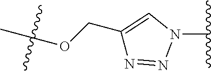 [SMILES: *OCc1cn(*)nn1]

Y_{b} is independently selected from:

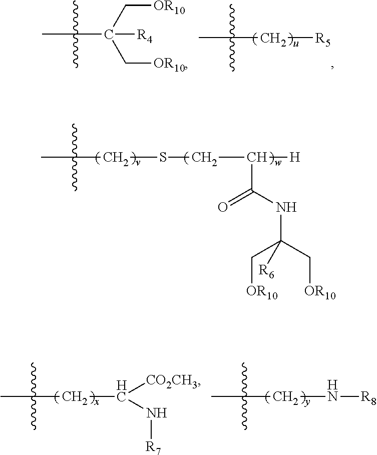

V is:

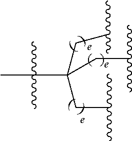

R₄, R₆ are each independently selected from H, C₁-C₆ alkyl or CH₂OR₁₀;
R₅ is a mono-, oligo-, polysaccharide or a cyclodextrin residue;
R₇, R₈ are each independently a peptide residue;
R₁₀ is H or a monosaccharide selected from glucose, galactose or mannose;
is 0 or 1;
j is 0 or 1;
e is 0, 1, 2, 3 or 4;
k is an integer from 1 to 12, preferably from 1 to 5;
r is an integer from 1 to 10;
u is 0, 1, 2, 3 or 4;
v is 1, 2, or 3;
w is an integer from 1 to 20, preferably from 1 to 10;
x, y are each independently an integer from 1 to 6.

In a particular embodiment, there are included dendrimers having the following formula:

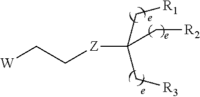

wherein
   W is R_{F} or a group selected from:

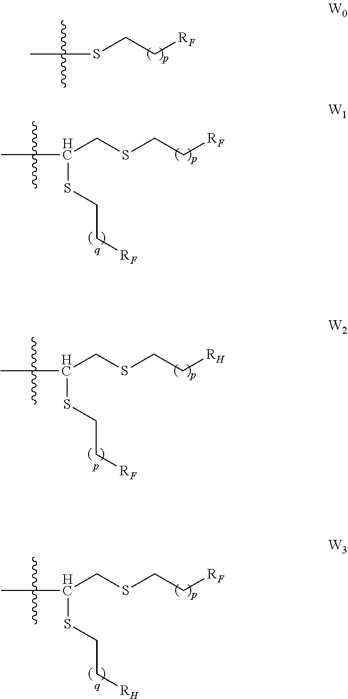

R_{F} being a C₁-C₂₄ perfluoroalkyl group, and R_{H} being a C₁-C₂₄ alkyl group,
p is 0, 1, 2, 3 or 4;
q is 0, 1, 2, 3 or 4;
Z is (CO)NH or NH(CO);
R₁, R₂, R₃ are H, or a group selected from (c) or (d):

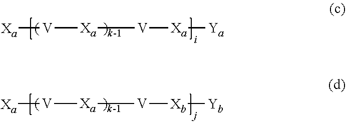

provided that:
R₁, R₂, R₃ are the same and selected from either group (c) or (d) or:
one of R₁, R₂, R₃ is H, the two others being the same and selected from either group (c) or (d);
X is Xₐ when j is 1 and X_{b} when j is 0;
Xₐ is at each occurrence independently selected from —OC(═O)CH₂—NH—, —OC(═O)CH₂—O—CH₂—, —O(CH₂)ᵣC(═O)—NH—, —O(CH₂)ᵣC(═O)—O—CH₂, OC(═O)NH—, —C(═O)—, —NH—, and —OCH₂—;
Yₐ is:

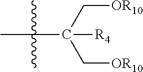

X_{b} is

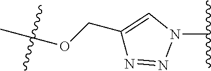 [SMILES: *OCc1cn(*)nn1]

Y_{b} is independently selected from:

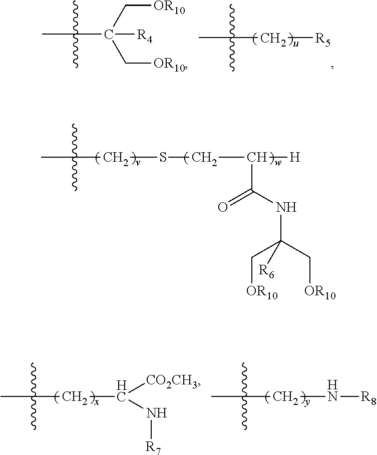

V is:

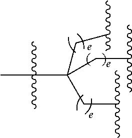

R₄, R₆ are each independently selected from H, C₁-C₆ alkyl or CH₂OR₁₀;
R₅ is a mono-, oligo-, polysaccharide or a cyclodextrin residue;
R₇, R₈ are each independently a peptide residue; R₁₀ is H or a monosaccharide selected from glucose, galactose or mannose;
is 0 or 1;
j is 0 or 1;
e is 0, 1, 2, 3 or 4;
k is an integer from 1 to 12, preferably from 1 to 5;
r is an integer from 1 to 10;
u is 0, 1, 2, 3 or 4;
v is 1, 2, or 3;
w is an integer from 1 to 20, preferably from 1 to 10;
x, y are each independently an integer from 1 to 6.

In another particular embodiment, R_{F} is a C₄-C₁₀ alkyl group.

In a particular embodiment, the hydrophilic terminal group of the surfactants defined above is of following formula:

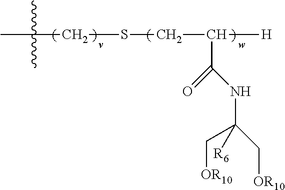

wherein R₆, R₁₀, v and w are as defined above, v being in particular equal to 3.

In a particular embodiment, the hydrophilic terminal group of the surfactants defined above is of following formula:

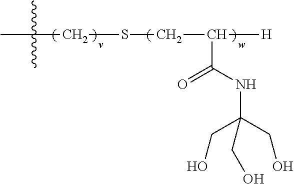

wherein v and w are as defined above, v being in particular equal to 3.

Suitable examples of amphiphilic dendrimers (Dendri-TAC) and preparation thereof are described in WO2016185425, and include F₆DiTAC₁₁, F₆DiTAC₆, F₆DiTAC₁₅, F₈DiTAC₅, DiF₆DiTAC₇, DiF₆DiTAC₁₅, DiF₈DiTAC₅, DiF₈DiTAC₁₁, having the following formulas:

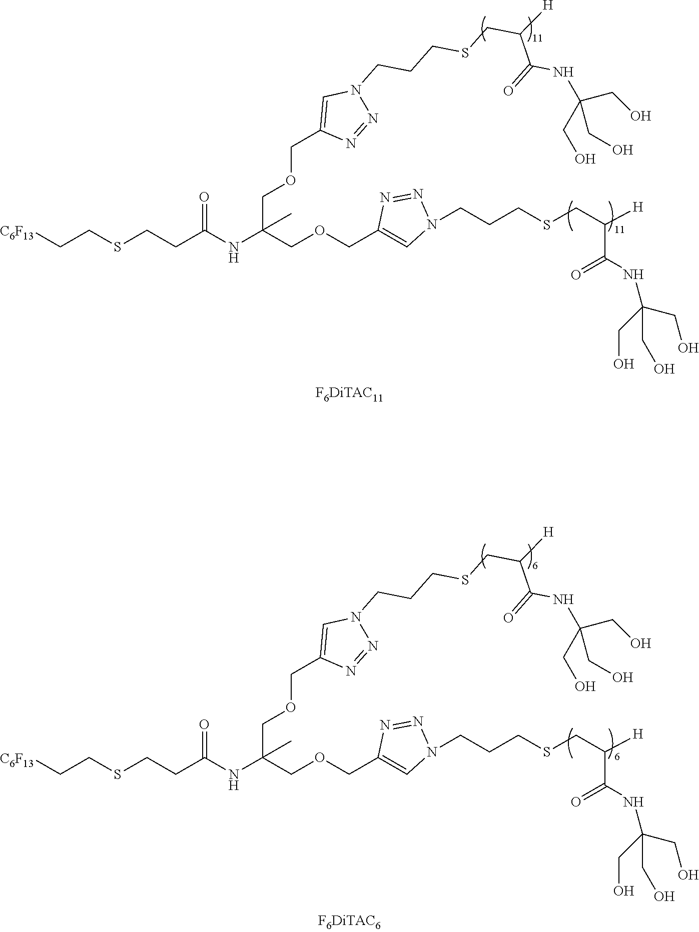

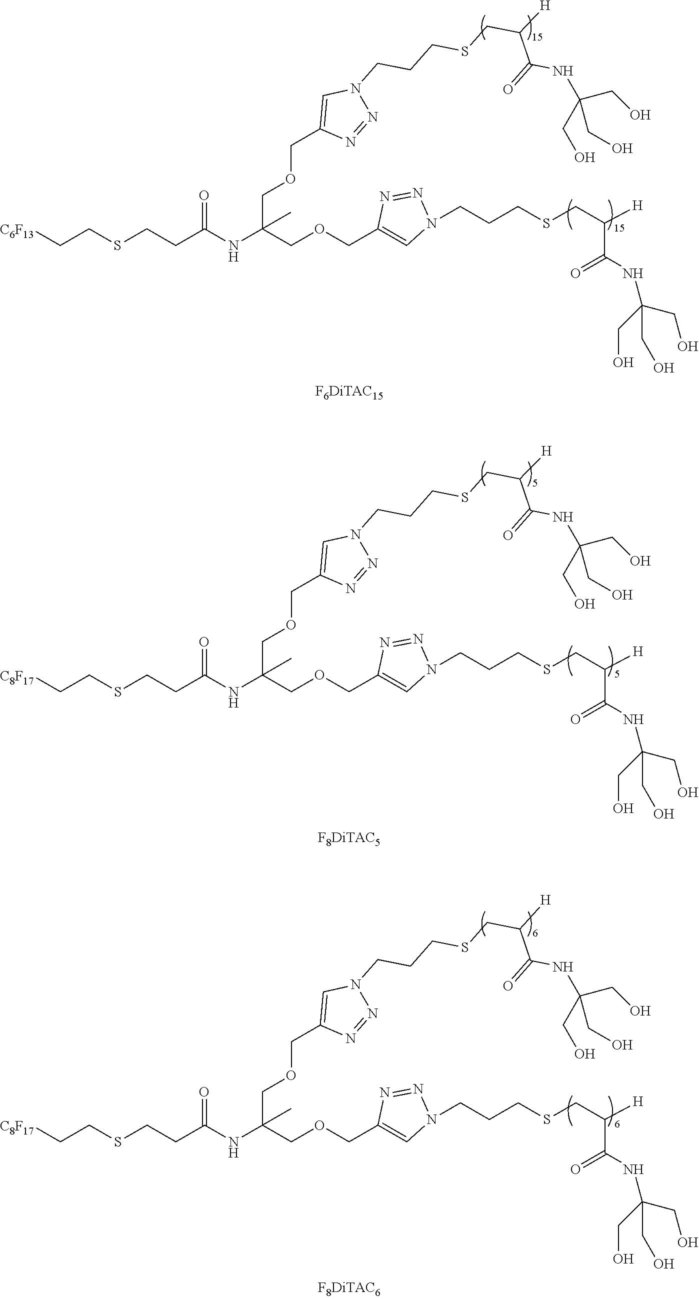

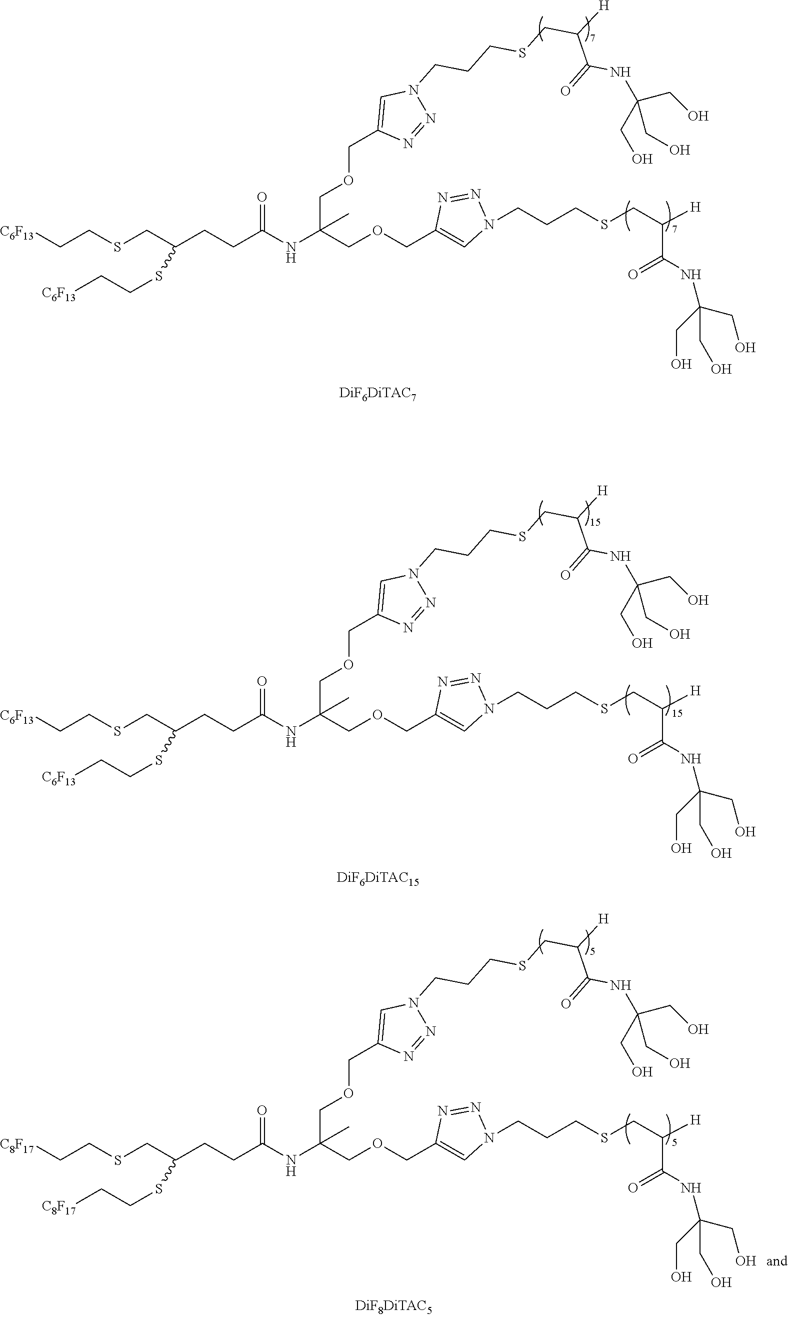

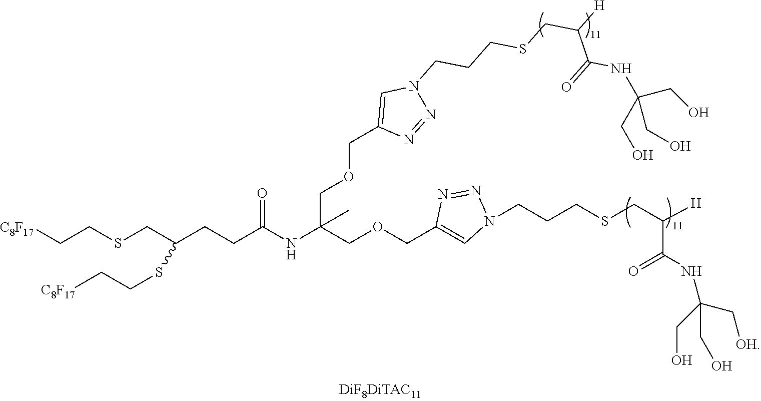

In a preferred embodiment of the present invention, the amphiphilic dendrimers Dendri-TAC are selected from the group comprising the following compounds of formula IA

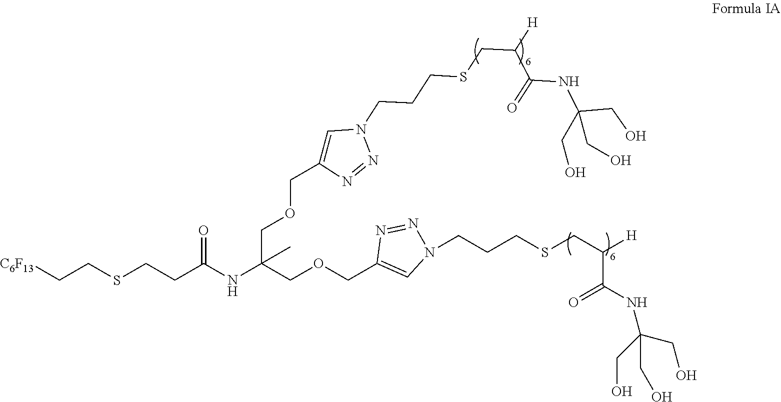

and of formula IB

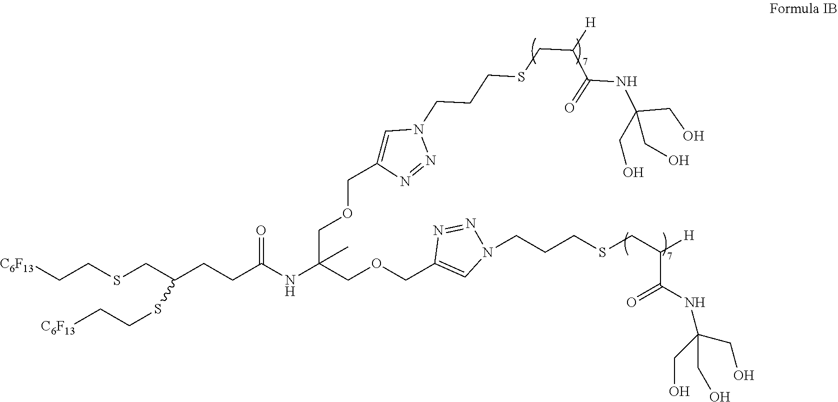

wherein the compound of formula IA is F₈DiTAC₆ and the compound of formula IB is DiF₆DiTAC₇.

In a preferred embodiment, the amphiphilic dendrimers Dendri-TAC is DiF₆DiTAC₇.

F-TACs comprise a hydrophilic part comprising an oligomer of polyTRIS type, and a hydrophobic part comprising a linear fluorinated alkyl chain.

In the present description and claims, the term F-TAC refers to a linear fluorinated surfactant having formula II:

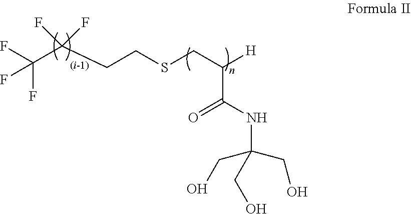

wherein:
n is the number of repeating Tris units (n=DPn is the average degree of polymerization), wherein the term Tris indicates the tris(hydroxymethyl)aminomethane unit, and
i is the number of carbon atoms in the fluoroalkyl chain.

In the present description and claims the compound of formula II can be interchangeably indicated as FᵢTACₙ, wherein:
n is the number of repeating Tris units (n=DPn is the average degree of polymerization) and
i is the number of carbon atoms in the fluoroalkyl chain.

According to an embodiment, i is comprised between 4 and 12, preferably between 6 and 10.

According to a further embodiment, when i is between 6 and 10, n is between 1 and 40, preferably between 4 and 30.

According to a still further embodiment, when i is 8, n is between 1 and 40, for example between 4 and 30.

Suitable examples of amphiphilic linear oligomers F-TAC have been disclosed for instance in Astafyeva, 2015, and include F₈TAC₇, F₈TAC₁₉, F₈TAC₁₈, F₈TAC₁₃ and F₆TAC₈ having the following formulas:

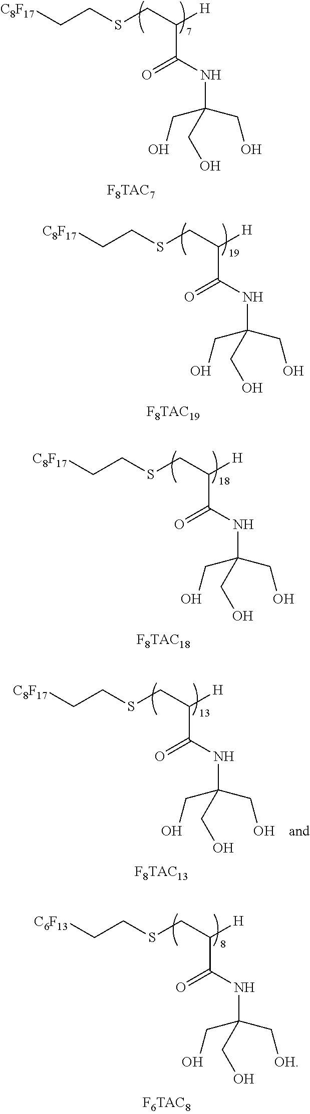

In an embodiment of the present invention, the amphiphilic linear oligomers (F-TAC) are selected from the group comprising the following compounds of formula IIA

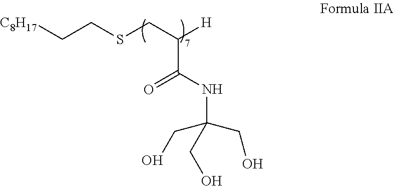

and of formula IIB

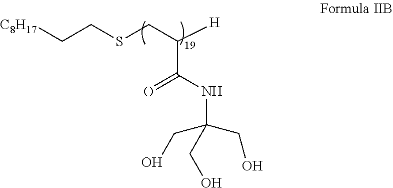

wherein the compound of formula IIA is F₈TAC₇ and the compound of formula IIB is F₈TAC₁₉.

The Applicants have now found that the physicochemical characteristics of the biocompatible fluorinated surfactants of the invention can influence the sizes of the disclosed (P)FC-NDs.

Table 1 shows selected physicochemical properties of preferred biocompatible fluorinated surfactants. In particular the values of surface tension, critical micelle concentration and molecular weight have been reported.

TABLE 1
Physicochemical properties of biocompatible
fluorinated surfactants

| Biocompatible fluorinated | ST | CMC | MW |
|---|---|---|---|
| surfactants | (mN/m) | (mmol/L) | (g/mol) |
| F₈DiTAC₆ (Formula IA) | 46.9 | 1.33E−02 | 2465 |
| DIF₆DiTAC₇ (Formula IB) | 41.9 | 2.42E−03 | 3471 |
| F₈TAC₇ (Formula IIA) | 32.3 | 1.34E−02 | 1705 |
| F₈TAC₁₉ (Formula IIB) | 40.9 | 2.70E−02 | 3630 |

ST: surface tension at 25° C.;
CMC: critical micelle concentration;
MW: molecular weight

For instance, in general it has been observed that the lower the surface tension value associated to the biocompatible fluorinated surfactant, the smaller the NDs size.

In the present description and claims, the expression surface tension has its common meaning in the chemistry field and indicates the tendency of liquid surfaces to shrink into the minimum surface area possible. Surfactants, such as the disclosed biocompatible fluorinated surfactants, are compounds that lower the surface tension between two liquids, between a gas and a liquid, or between a liquid and a solid.

For instance, surface tension can be measured using the Wilhelmy plate technique, e.g. at the air/water interface, at (25.0±0.5) ° C. with a K100 tensiometer (Kruss, Hamburg, Germany).

According to an embodiment, preferred biocompatible fluorinated surfactants are characterized by a surface tension value lower than 70 mN/m, more preferably lower than 50 mN/m.

(Per)fluorocarbons

In the present description and claims the term “fluorocarbons” refers to a group of fluorine-containing compounds derived from hydrocarbons by partial or complete substitution of hydrogen atoms with fluorine atoms, which are liquid at room temperature. Preferably the fluorocarbon is a perfluorocarbon (PFC), i.e. a fluorinated hydrocarbon where all the hydrogen atoms are substituted with fluorine atoms.

Liquid (per)fluorocarbons are characterized by a boiling point comprised between 25° C. and 160° C. In the present invention, the (per)fluorocarbons are preferably characterized by a boiling point comprised between 25° C. and 100° C., still more preferably between 27° C. and 60° C.

Suitable examples of fluorocarbons are 1-Fluorobutane, 2-Fluorobutane, 2,2-Difluorobutane, 2,2,3,3-Tetrafluorobutane, 1,1,1,3,3-Pentafluorobutane, 1,1,1,4,4,4-Hexafluorobutane, 1,1,1,2,4,4,4-Heptafluorobutane, 1,1,2,2,3,3,4,4-Octafluorobutane, 1,1,1,2,2-Pentafluoropentane, 1,1,1,2,2,3,3,4-Octafluoropentane, 1,1,1,2,2,3,4,5,5,5-Decafluoropentane, 1,1,2,2,3,3,4,4,5,5,6,6-Dodecafluorohexane.

Suitable examples of perfluorocarbons are perfluoropentane, perfluorohexane, perlfluoroheptane, perfluorooctane, perfluorononane, perfluorodecalin, perfluorooctylbromide (PFOB), perfluoro-15-crown-5-ether (PFCE), perfluorodichlorooctane (PFDCO), perfluorotributylamine (PFTBA), perfluorononane (PFN), and 1,1,1-tris(perfluorotert-butoxymethyl)ethane (TPFBME), or a mixture thereof.

In an embodiment said perfluorocarbon is preferably perfluoropentane (PFP) (boiling point 29° C.), perfluorohexane (PFH) (boiling point 57° C.) or perfluorooctylbromide (PFOB) (boiling point 142° C.).

BFS/(Per)Fluorocarbons Molar Ratio

In the present description and claims the expression “molar ratio” (Nr) indicates the ratio of biocompatible fluorinated surfactant and (per)fluorocarbon ((P)FC) that is used to stabilize the inner core of the disclosed nanodroplets. It is possible to calculate the molar ratio by using the following formula:

$$Molar ratio Nr = \frac{total moles of biocompatible fluorinated surfactant}{total moles of ( P ) FC} \qquad Eq . 1$$

wherein:
   the expression “total moles of biocompatible fluorinated surfactant” indicate the molar amount of biocompatible fluorinated surfactants in the nanodroplets suspension, and
   the expression “total moles of (P)FC” indicate the molar amount of (per)fluorocarbons forming the inner core of the NDs.

In general, the molar amount of biocompatible fluorinated surfactants in the NDs suspension refers to the molar amount of biocompatible fluorinated surfactants forming the outer layer of the nanodroplets or the molar amount of the biocompatible fluorinated surfactants not bound to the stabilizing layer (e.g. in free or micellar form in the aqueous suspension), or both.

In an embodiment, the molar amount of biocompatible fluorinated surfactant into the aqueous phase ranges between 0.0006 and 0.006 mmol, preferably between 0.002 and 0.004 mmol.

In an embodiment, the molar amount of (P)FC into the organic phase ranges between 0.01 and 0.04 mmol, preferably between 0.014 and 0.028 mmol.

An aspect of the present invention relates to a nanodroplet comprising an outer layer and an inner core wherein said outer layer comprise a biocompatible fluorinated surfactant and said inner core comprises a fluorocarbon wherein the molar ratio between said biocompatible fluorinated surfactant and said fluorocarbon is higher than 0.06.

In an embodiment the molar ratio between said biocompatible fluorinated surfactant and said fluorocarbon is higher than 0.060, preferably higher than 0.068, preferably higher than 0.070, preferably higher than 0.080, preferably higher than 0.090, preferably higher than 0.100, preferably higher than 0.140 and still more preferably is higher than 0.190.

On the other side, the Applicants have observed that the molar ratio between said biocompatible fluorinated surfactant and said fluorocarbon should preferably not be higher than 0.300, more preferably not higher than 0.250.

Calibrated (Per)Fluorocarbon Nanodroplets

A further aspect relates to an aqueous suspension comprising a nanodroplet as above defined.

A preferred embodiment relates to an aqueous suspension comprising a plurality of nanodroplets as above defined, wherein said nanodroplets have a z-average diameter comprised between 100 nm and 1000 nm and a polydispersity lower than 0.25.

In the present description and claims, the term “plurality of nanodroplets” refers to a population of nanodroplets characterized by a calibrated distribution, meaning that substantially all the nanodroplets have substantially similar sizes.

The expression “calibrated distribution” indicates a polydispersity (PDI) of a certain population of nanodroplets (e.g. with a z-average diameter comprised between 100 and 1000 nm) with a polydispersity index (PDI) lower than 0.25, preferably lower than 0.2, more preferably lower than 0.15, even more preferably lower than 0.1.

The term “polydispersity” (PDI) refers to a dimensionless measure of the broadness of the size distribution calculated from the cumulants analysis, wherein said cumulants analysis, defined in the International Standard on Dynamic Light Scattering ISO13321 (1996) and ISO22412 (2008), gives a mean particle size (z-average) and an estimate of the width of the distribution (polydispersity index).

For instance, a polydispersity higher than 0.7 indicates a very broad distribution of particles sizes, while a value lower than 0.08 indicates a nearly monodisperse sample characterized by a monomodal distribution. The polydispersity can be measured with the dynamic light scattering technique (DLS), by using for instance the Malvern Zetasizer Nano-ZS instrument (Malvern Instruments Ltd., UK).

The “z-Average Diameter (ZD)” is defined as the intensity-weighted mean diameter derived from the cumulants analysis. In other words, it relates to the average of calibrated nanodroplets size dispersed in the aqueous suspension measured through the dynamic light scattering technique (DLS).

In the present invention, the z-average diameter is comprised between 100 nm and 1000 nm, preferably between 120 and 600, more preferably between 150 and 400.

Suitable aqueous carriers for the aqueous suspension of the present invention, which are preferably physiologically acceptable, comprise water (preferably sterile water), aqueous solutions such as saline (which may advantageously be balanced so that the final product for injection is not hypotonic), or solutions of one or more tonicity adjusting substances. Tonicity adjusting substances comprise salts or sugars, sugar alcohols, glycols or other non-ionic polyol materials (e.g. glucose, sucrose, trehalose, sorbitol, mannitol, glycerol, polyethylene glycols, propylene glycols and the like), chitosan derivatives, such as carboxymethyl chitosan, trimethyl chitosan or gelifying compounds, such as carboxymethylcellulose, hydroxyethyl starch or dextran.

In the present invention, calibrated (per)fluorocarbon nanodroplets are preferably produced by using a microfluidic technology.

Stability

The Applicants have now surprisingly found that it is possible to substantially improve the stability of the disclosed calibrated (per)fluorocarbon nanodroplets by tuning the molar ratio between the biocompatible fluorinated surfactant and the fluorocarbon.

In the present description and claims, the term “stability” indicates the property of a nanodroplets composition to substantially maintain over time its initial NDs sizes and preferably also its initial monodispersed distribution.

Initial NDs sizes and initial monodispersed distribution refer to the values of NDs sizes and monodispersity of the calibrated NDs composition at the end of the preparation process.

For the sake of clarity, end of the preparation process refers either to i) the collection of the calibrated NDs from the exit channel of the microfluidic cartridge or ii) the collection of said calibrated NDs followed by a dilution step (i.e. step e).

Both these alternative final stages are performed before any storage period of the calibrated NDs.

The expression “storage period” indicates a period of time, e.g. expressed as hours, days or weeks, during which a microfluidically-prepared aqueous suspension of calibrated NDs is kept under certain conditions of temperature after the end of the preparation process.

It is possible to calculate the stability of the calibrated (per)fluorocarbon nanodroplets by using the NDs size evolution (% Evol) parameter, according to the following equation (Eq.2):

$$\% Evol = \frac{( Dfinal - Dinitial )}{Dinitial} \times 100 \qquad Eq . 2$$

### Wherein:

D final is the z-average diameter of the calibrated NDs after a certain time from the end of the preparation process, e.g. after 60 minutes from the end of preparation process or after a storage period (e.g. one week) at different conditions (different temperatures, pressure, etc); and
D initial is the z-average diameter of the calibrated NDs immediately (e.g. within minutes) at the end of its preparation process.

In the present invention, a value of % Evol close to 0 (either positive or negative) indicates a higher stability of the calibrated NDs suspension, whereby the nanodroplets in the suspension substantially maintain over time their initial mean dimensions.

According to the present invention, the % Evol of a calibrated NDs suspension is preferably lower than +50%, more preferably lower than +30%, and still more preferably is lower than +20%.

The Applicants have unexpectedly found that increasing the molar ratio between BFS and (per)fluorocarbon allowed to reduce the % Evol and thus increase the stability of the disclosed calibrated (per)fluorocarbon NDs stabilized by BFS.

The Applicants found that the % Evol parameter has a significant dependence from the molar ratio between BFS and fluorocarbon. In particular, this effect is more evident with higher molar ratio values, which is preferably higher than 0.08, more preferably higher than 0.1 and still more preferably is higher than 0.14.

Moreover, it has been observed that an increased molar ratio between BFS and fluorocarbon has also a substantially positive influence on maintaining the PDI values over time.

Microfluidic Cartridge

The NDs of the present invention are preferably produced through a bottom-up approach using a microfluidic technique.

In the present description and claims the expression “microfluidic technique” refers to a technology of manufacturing nanodroplets through a microfluidic cartridge designed to manipulate fluids in channels at the microscale.

Said microfluidic technique is a bottom-up approach, that is to say that the nanodroplets are obtained by assembling molecules (e.g. BFS and (per)fluorocarbons) into larger nanostructures (i.e. nanodroplets).

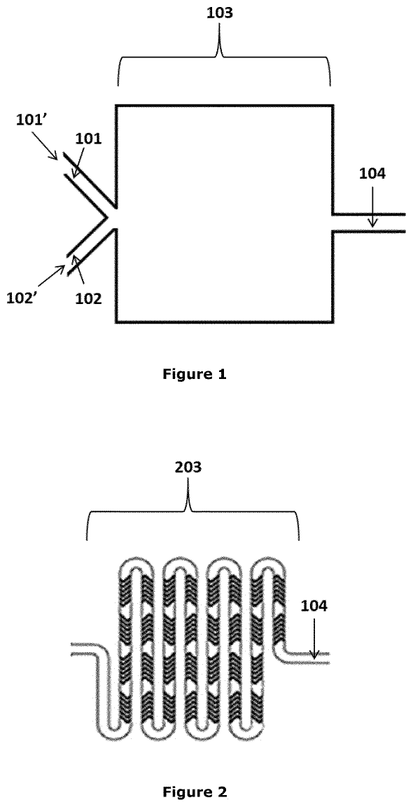

FIG. 1 shows a schematic representation of the core portion 100 of a microfluidic cartridge (i.e., microfluidic cartridge) useful in the process of the invention. The cartridge comprises a first inlet 101 for feeding the aqueous phase 101′ and a second inlet 102 for supplying the organic phase 102′. The aqueous phase and the organic phase are directed towards a mixing device 103, for instance a staggered herringbone micromixer 203 as illustrated in FIG. 2, wherein they are mixed (e.g. through laminar mixing in the case of the micromixer of FIG. 2 endowing to the formation of NDs.

The calibrated fluorocarbon NDs are then directed to the exit channel 104, from where they are collected in a suitable container (e.g. a vial).

Alternatively, said microfluidic cartridge can be equipped with an additional channel, for instance placed between the mixing device 103 and the exit channel 104, aimed at diluting, with a suitable solvent, the calibrated fluorocarbon NDs suspension before their direction to the exit channel 104 (i.e. in-line dilution).

A mixing device 103 is generally characterized by suitable geometries able to enhance the microfluidic-mixing performance. In fact, the mixing process takes place into the peculiar micro-channel geometry of the mixing device, which causes fluid streams to mix together on the way to exit the microfluidic cartridge.

Different types of mixing devices are available with different shapes or microstructures. Suitable examples of mixing devices can be classified as passive micromixers, such as T and Y shaped mixers (e.g. staggered herringbone micromixer or toroidal mixer), and the mixer using flow focusing; and active micromixers, such as mixer using pressure field disturbance, electrokinetic active micromixer and ultrasound active micromixer.

Preferred in the present invention is a staggered herringbone micromixer (FIG. 2), wherein the mixing of the two liquid phases is controlled by lamination-mixing or a toroidal micromixer.

During the mixing phase the (per)fluorocarbon nanodroplets are formed and directed to the exit channel of the microfluidic cartridge, or, alternatively, directed to an additional channel to dilute the nanodroplets before their direction to the exit channel.

In the present description and claims, the expression “exit (or outlet) channel” indicates the terminal portion of the microfluidic cartridge, toward which the just formed nanodroplets are directed from the mixing device and from where it is possible to collect the formed suspension of nanodroplets in a suitable container (e.g. a vial).

An example of microfluidic cartridge is the commercially available NxGen Cartridge, with or without in-line dilution, from Precision Nanosystems (Vancouver, Canada). These microfluidic cartridges can comprise either staggered herringbone or toroidal micromixers, both operating under non-turbulent conditions. For the manufacturing process, the microfluidic cartridge is mounted on a microfluidic instrument, generally equipped with a cartridge adapter, to host the microfluidic cartridge, and with containers (e.g. syringes or vials for continuous-flow injection) directly connected to the inlets of the microfluidic cartridge and specifically designed to pump the liquid phases into said inlets. Example of a microfluidic instrument is the NanoAssemblr® Benchtop Automated Instrument (Precision Nanosystems (Vancouver, Canada)).

An aspect of the invention relates to a method for the preparation of an aqueous suspension of calibrated fluorocarbon nanodroplets, said method comprising the steps of:
c) Preparing an aqueous phase;
d) Preparing an organic phase,
wherein i) said aqueous phase comprises a biocompatible fluorinated surfactant selected from Dendri-TAC, F-TAC or a mixture thereof and the organic phase comprises a fluorocarbon or
ii) said organic phase comprises a biocompatible fluorinated surfactant selected from Dendri-TAC, F-TAC or a mixture thereof and a fluorocarbon.
c) Injecting said aqueous phase into a first inlet and said organic phase into a second inlet of a microfluidic cartridge, thereby mixing said aqueous phase and said organic phase in a mixing device of the microfluidic cartridge to obtain an aqueous suspension of calibrated fluorocarbon nanodroplets, and
d) Collecting the aqueous suspension of calibrated fluorocarbon nanodroplets from the exit channel of the microfluidic cartridge.

According to a preferred embodiment, said aqueous phase comprises a biocompatible fluorinated surfactant selected from Dendri-TAC, F-TAC or a mixture thereof and said organic phase comprises a fluorocarbon.

Preferably said fluorocarbon is a perfluorocarbon.

Typically, at step c) the injection of the aqueous phase and the injection of the organic phase are carried out simultaneously.

The expression “simultaneously” indicates the simultaneous injection (i.e. co-injection) of the aqueous phase and the organic phase into the microfluidic cartridge, that is to say that the aqueous phase and organic phase are injected into two separate inlets of the microfluidic cartridge at the same time or at substantially the same time (e.g. within few seconds).

According to the disclosed method it is possible to obtain an aqueous suspension of calibrated (per)fluorocarbon nanodroplets by a single passage of the liquid phases through the two-channel microfluidic system.

In an embodiment said method for the preparation of an aqueous suspension of calibrated (per)fluorocarbon nanodroplets is the microfluidic technique, wherein said calibrated (per)fluorocarbon nanodroplets (Z-average diameter comprised between 100 and 1000 nm) have a polydispersity index (PDI) lower than 0.25, preferably lower than 0.20, more preferably lower than 0.15, even more preferably lower than 0.1.

Advantageously, when the surfactant is dissolved in the aqueous phase, the present novel method can be used for the preparation of an aqueous-suspension of calibrated nanodroplets stabilized by any other biocompatible surfactants, particularly biocompatible fluorinated surfactants, other than Dendri-TAC and FTAC.

As mentioned above, the expression “biocompatible fluorinated surfactant” refers to amphiphilic organic compounds suitable for forming the stabilizing layer of a nanodroplet, comprising a hydrophilic moiety and a hydrophobic moiety. Said amphiphilic organic compounds have substantial compatibility with living tissue or a living system by not being toxic, injurious, or physiologically reactive and typically not causing immunological rejection.

A further aspect of the invention is thus related to a method for the preparation of an aqueous suspension of calibrated nanodroplets, said method comprising the steps of:
a) Preparing an aqueous phase comprising a biocompatible surfactant;
b) Preparing an organic phase comprising a fluorocarbon;
c) Injecting said aqueous phase into a first inlet and said organic phase into a second inlet of a microfluidic cartridge, thereby mixing said aqueous phase and said organic phase in a mixing device of the microfluidic cartridge to obtain an aqueous suspension of calibrated nanodroplets, and
d) Collecting the aqueous suspension of calibrated nanodroplets from the exit channel of the microfluidic cartridge.

Preferably said biocompatible surfactant is a biocompatible fluorinated surfactant.

Preferably said fluorocarbon is a perfluorocarbon.

Aqueous Phase

The expression “aqueous phase” refers to a liquid comprising an aqueous liquid component, including for instance water, aqueous buffered solutions, aqueous isotonic solutions or a mixture thereof. Preferably the aqueous phase is water.

According to a preferred embodiment, said aqueous phase comprises a biocompatible fluorinated surfactant selected from the amphiphilic linear oligomers F-TAC and the amphiphilic dendrimers Dendri-TAC, as described above, or a mixture thereof.

For instance, a biocompatible fluorinated surfactant can be admixed with an aqueous component through traditional techniques (e.g. stirring) in order to prepare the aqueous phase to be injected into the first inlet of the microfluidic cartridge.

At step a) the aqueous phase comprises a biocompatible fluorinated surfactant at a concentration ranging between 0.0006 mmol/mL and 0.006 mmol/mL, more preferably between 0.0001 mmol/mL to 0.015 mmol/mL, still more preferably between 0.001 mmol/mL and 0.01 mmol/mL.

Organic Phase

The expression “organic phase” refers to a liquid comprising an organic solvent miscible with water including methanol, ethanol, isopropanol, acetonitrile and acetone.

Preferably the organic phase is ethanol.

According to a preferred embodiment, said organic phase comprises a fluorocarbon or a mixture of different fluorocarbons. Preferably the fluorocarbons are perfluorocarbons.

Suitable examples of (per)fluorocarbons are those mentioned above.

As an example, a fluorocarbon can be admixed with an organic solvent through traditional techniques (e.g. stirring) in order to prepare the organic phase to be injected into the second inlet of the microfluidic cartridge In a further embodiment, at step b) the organic phase comprises a (per)fluorocarbon at a concentration ranging between 0.003 mmol/mL and 0.142 mmol/mL, more preferably between 0.011 mmole/mL and 0.085 mmole/mL, still more preferably between 0.013 mmol/mL to 0.057 mmol/mL.

In an alternative embodiment, said organic phase comprises a biocompatible fluorinated surfactant as defined above and a fluorocarbon.

For instance, said organic phase comprises
i) a biocompatible fluorinated surfactant at a concentration ranging between 0.0006 mmol/mL and 0.006 mmol/mL, more preferably between 0.0001 mmol/mL to 0.015 mmol/mL, still more preferably between 0.001 mmol/mL and 0.01 mmol/mL; and
ii) a (per)fluorocarbon at a concentration ranging between 0.003 mmol/mL to 0.142 mmol/mL, more preferably between 0.011 mmol/mL and 0.085 mmol/mL, still more preferably between 0.013 mmol/mL to 0.057 mmol/mL.

In a still further embodiment of the invention both the aqueous and organic phases are preferably injected at a temperature lower than room temperature (e.g. from about 4° C. to 20° C.) into the microfluidic cartridge, to avoid vaporization of fluorocarbons having a boiling point close to room temperature.

A further aspect relates to an aqueous suspension comprising a plurality of calibrated fluorocarbon nanodroplets obtainable by a method of preparation comprising the steps of:
a) Preparing an aqueous phase;
b) Preparing an organic phase,
wherein i) said aqueous phase comprises a biocompatible fluorinated surfactant, selected from Dendri-TAC, F-TAC or a mixture thereof, and the organic phase comprises a fluorocarbon or
ii) said organic phase comprises a biocompatible fluorinated surfactant, selected from selected from Dendri-TAC, F-TAC or a mixture thereof and a fluorocarbon.
c) Injecting said aqueous phase into a first inlet and said organic phase into a second inlet of a microfluidic cartridge, thereby mixing said aqueous phase and said organic phase in a mixing device of the microfluidic cartridge to obtain an aqueous suspension of calibrated fluorocarbon nanodroplets, and
d) Collecting the aqueous suspension of calibrated fluorocarbon nanodroplets from the exit channel of the microfluidic cartridge.

In a preferred embodiment, said aqueous phase comprises a biocompatible fluorinated surfactant selected from the selected from Dendri-TAC, F-TAC or a mixture thereof, and the organic phase comprises a fluorocarbon, being preferably a perfluorocarbon.

In a further embodiment said calibrated (per)fluorocarbon nanodroplets have a Z-average diameter comprised between 100 and 1000 nm and a polydispersity index (PDI) lower than 0.25, preferably lower than 0.20, more preferably lower than 0.15, even more preferably lower than 0.1

Total Flow Rate (TFR) and Flow Rate Ratio (FRR)

The method of the present invention allows to control the (per)fluorocarbon nanodroplets characteristics by varying two process parameters: the Total Flow Rate and the Flow Rate Ratio.

The expression “Total Flow Rate (TFR)” refers to the total flow of both fluid streams, namely the aqueous phase and the organic phase, being pumped through the two separate inlets of the microfluidic cartridge. The unit of measurement of the TFR is mL/min.

According to an embodiment, the TFR is preferably comprised between 2 mL/min and 18 mL/min, more preferably between 5 mL/min and 16 mL/min, still more preferably the TFR is 10 mL/min.

The expression “Flow Rate Ratio (FRR)” refers to the ratio between the amount of aqueous phase and the amount of organic phase flowing into the microfluidic cartridge, according to the Equation 3:

$$Flow rate ratio = \frac{v olume of aqueous phase}{v olume of organic phase} \qquad Eq . 3$$

The volume of aqueous and organic phases can be expressed as e.g. mL.

In a preferred embodiment, the FRR (volume of aqueous phase vs. volume of organic phase) is between 1:1 to 5:1, preferably between 1:1 and 3:1, more preferably the FRR is 1:1.

In the present invention, the respective concentrations of both biocompatible fluorinated surfactant and fluorocarbon and the FRR can be purposely tuned in order to obtain a molar ratio between said biocompatible fluorinated surfactant and said fluorocarbon higher than 0.060, preferably higher than 0.068, preferably higher than 0.070, preferably higher than 0.080, preferably higher than 0.090, preferably higher than 0.100, preferably higher than 0.140 and still more preferably is higher than 0.190.

The molar ratio between said biocompatible fluorinated surfactant and said fluorocarbon should preferably not be higher than 0.300, more preferably not higher than 0.250

Optional Step e) Dilution

According to present invention, the method of preparation further comprises an optional step e), which comprises diluting the collected aqueous suspension of calibrated fluorocarbon nanodroplets.

The Applicants have unexpectedly observed that a dilution step after the NDs production, using a microfluidic cartridge, has a favorable effect on the initial NDs size and initial monodispersity. Indeed, without dilution, the NDs size was larger than with a dilution.

As indicated above, the expressions “initial monodispersed distribution” and “initial NDs sizes” refer to the values of monodispersity and NDs sizes of the calibrated NDs composition at the end of its preparation process, wherein said end of the preparation process refers either to i) the collection of the calibrated NDs from the exit channel of the microfluidic cartridge or ii) the collection of said calibrated NDs followed by a dilution step (i.e. step e).

In the present description and claims the term “dilution” refers to the process of reducing the concentration of calibrated nanodroplets in the suspension, by adding a suitable amount of aqueous liquid, including water or an aqueous solution.

A suitable amount of aqueous liquid corresponds to the quantity of aqueous liquid necessary to reduce the concentration of the calibrated nanodroplets in the aqueous suspension from 2 to 10-folds.

In a preferred embodiment, the optional step e) of the present method comprises diluting the collected aqueous suspension of calibrated fluorocarbon nanodroplets from 1 to 20-folds, preferably from 3 to 8-folds, still more preferably the collected aqueous suspension is diluted 5-fold.

An additional effect of dilution is that of reducing the relative amount of organic solvent in the suspension.

In a further preferred embodiment, the step e) of the present method comprises diluting the collected suspension of calibrated fluorocarbon with water.

As mentioned above, the diluting step can be alternatively performed inside the microfluidic cartridge, due to an additional channel (e.g. placed between the mixing device 103 and the exit channel 104 in FIG. 1) aimed at diluting the calibrated fluorocarbon NDs suspension before their direction to the exit channel.

In this case, the step e) of the present method comprises diluting the suspension of calibrated fluorocarbon nanodroplets from 1 to 20-folds, preferably from 3 to 8-folds, still more preferably the collected aqueous suspension is diluted 5-fold, before their collection from the microfluidic cartridge.

Optional Step f): Freezing

A still further embodiment of the invention relates to a method for the preparation of an aqueous suspension of calibrated fluorocarbon nanodroplets, comprising an optional step f), subsequent to step e), comprising freezing the suspension of calibrated (per)fluorocarbon nanodroplets.

In an embodiment, said step f) comprises freezing the suspension of calibrated (per)fluorocarbon nanodroplets at a temperature comprised between −60° C. and 0° C., preferably comprised between −40° C. and −10° C., still more preferably the temperature is −30° C.

The frozen suspension can then be stored at a temperature comprised between −30° and −10° C., preferably −20° C.

The Applicants observed that freezing the suspension of calibrated NDs suspensions allowed further decreasing the % Evol, independently from the used molar ratios.

In a further embodiment, said step f) comprises freezing the calibrated (per)fluorocarbon nanodroplet for a time comprised between 1 and 60 minutes, preferably comprised between 5 and 30 minutes, still more preferably the time is 15 minutes.

In a further embodiment, before the optional step f), the dilution at step e) is performed using an aqueous solution, as mentioned above, comprising at least a cryoprotectant agent.

The expression “cryoprotectant agent” designates any compound able to improve the freezing efficiency maintaining the initial NDs sizes and to substantially maintaining over time their initial monodispersed distribution and their initial NDs sizes. Examples of components suitable for stabilizing the calibrated nanodroplets are polyethylene glycols (PEG), polyols, saccharides, surfactants, buffers, amino acids, chelating complexes, and inorganic salts. Preferably, the component suitable for stabilizing the calibrated nanodroplets is a saccharide.

Preferably, said saccharide is selected from the group of disaccharides, trisaccharides and polysaccharides, more preferably is a disaccharide. Examples of disaccharides include: trehalose, maltose, lactose, and sucrose. Particularly preferred among the disaccharides is trehalose.

In a preferred embodiment, the aqueous solution at step d) comprises trehalose.

In a still preferred embodiment, said aqueous solution has a concentration of trehalose comprised between 1 to 10%, preferably 3 to 7%, more preferably is 5%.

An aspect of the invention relates to an aqueous suspension comprising a nanodroplet as above defined and trehalose.

Another aspect of the invention refers to an aqueous suspension comprising a plurality of nanodroplets as above defined, wherein said nanodroplets have a polydispersity index (PDI) lower than 0.25, preferably lower than 0.20, more preferably lower than 0.15, even more preferably lower than 0.10, and a Z-average diameter comprised between 100 nm and 1000 nm, preferably between 120 and 600 nm, more preferably between 150 and 400 nm, and trehalose.

Acoustic Droplet Vaporization

The expression “acoustic droplet vaporization (ADV)” refers to the phase-shift of the inner core of a (per)fluorocarbon nanodroplet from liquid to gas state as a result of an applied ultrasound energy beyond a vaporization threshold. Said ultrasound act as an external stimulus to promote the vaporization of the droplets in a controllable, non-invasive and localized manner.

Below vaporization threshold, the nanodroplets are ultrasonically stable with low acoustic attenuation and can be acoustically vaporized at the location of interest. Thanks to their smaller size and volume compared to conventional microbubbles, nanodroplets display prolonged in vivo circulation, deep penetration into the tissues via the extravascular space (Helfield et al. 2020).

Use

Calibrated BFS-PFC nanodroplets can represent an alternative to gaseous microbubbles for medical ultrasound applications. Upon applying ultrasound energy, droplets can be selectively vaporized in a region of interest to form microbubbles. After activation, the calibrated BFS-PFC nanodroplets can substantially be used in the same manner as conventional Contrast-Enhanced UltraSound (CEUS).

An aspect relates to an aqueous suspension obtained according to the process as above defined for use in a diagnostic and/or therapeutic treatment.

A further aspect relates to an aqueous suspension comprising a nanodroplet or a plurality of nanodroplets as above defined and trehalose for use in a diagnostic and/or therapeutic treatment.

Diagnostic treatment includes any method where the use of the gas-filled microvesicles allows enhancing the visualisation of a portion or of a part of an animal (including humans) body, including imaging for preclinical and clinical research. Suitable examples of diagnostic applications are molecular and perfusion imaging, tumor imaging (EPR effect), multimodal imaging (MR-guided tumor ablation, fluorescence, sono-photoacoustic activation), US aberration correction and super resolution ultrasound imaging.

Therapeutic treatment includes any method of treatment of a patient. In preferred embodiments, the treatment comprises the combined use of ultrasounds and (per)fluorocarbons nanodroplets either as such (e.g. in ultrasound mediated thrombolysis, high intensity focused ultrasound ablation, blood-brain barrier permeabilization, immunomodulation, neuromodulation, radiosensitization) or in combination with a therapeutic agent (i.e. ultrasound mediated delivery, e.g. for the delivery of a drug or bioactive compound to a selected site or tissue, such as in tumor treatment, gene therapy, infectious diseases therapy, metabolic diseases therapy, chronic diseases therapy, degenerative diseases therapy, inflammatory diseases therapy, immunologic or autoimmune diseases therapy or in the use as vaccine), whereby the presence of the nanodroplets may provide a therapeutic effect itself or is capable of enhancing the therapeutic effects of the applied ultrasounds, e.g. by exerting or being responsible to exert a biological effect in vitro and/or in vivo, either by itself or upon specific activation by various physical methods (including e.g. ultrasound mediated delivery).

The following examples will help to further illustrate the invention.

### EXAMPLES

Materials and Methods

The following materials are employed in the subsequent examples:

| | |
|---|---|
| F₈DiTAC₆ | Formula IA |
| DiF₆DiTAC₇ | Formula IB |
| F₈TAC₇ | Formula IIA |
| F₈TAC₁₉ | Formula IIB |
| Perfluoropentane | 288 g/mol, bp: 28-30° C. - |
| (Dodecafluoropentane) | Interchim (Montluçon, France) |
| Perfluorohexane | 338 g/mol, bp; 58-60° C. - |
| (Tetradecafluorohexane) | Aldrich (United Kingdom) |
| PFOB (Perfluorooctylbromide) | 498.96 g/mol, bp: 142° C. - |
| | Sigma-Aldrich |
| Ethanol | 96% Thommen-Furler AG |
| D-(+)-trehalose dihydrate | Sigma (USA) |
| (378.3 g/mol) | |

### Example 1

Preparation of PFC Nanodroplets Using a Microfluidic Platform

Perfluorocarbon nanodroplets were formulated with a NanoAssembr™ Benchtop automated instrument from Precision Nanosystems (Vancouver, Canada) equipped with a staggered herringbone micromixer (SHM) allowing size-controlled self-assemblies. Briefly, an aqueous phase comprising a biocompatible fluorinated surfactant (BFS) was injected into the first inlet whereas the organic phase composed of PFC dissolved in ethanol was injected into the second inlet of the microfluidic cartridge (FIG. 1). Both phases were placed into an ice bath at about 4° C. before the NDs formulation. Microscopic characteristics of the channels are engineered to cause an accelerated mixing of the two fluid streams in a controlled fashion. The microfluidic process settings namely the Total Flow Rate (TFR, in mL/min), and the Flow Rate Ratio (FRR), were varied to control the NDs characteristics. The NDs suspensions were collected from the exit channel in a Falcon vial (15 mL).

Alternatively (see example 5), the collected NDs suspension was diluted with ultrapure water or trehalose solution (5% final).

### Example 2

Effects of the BFS/PFC Molar Ratios

The effects of BFS/PFC molar ratios on the stability of the calibrated perfluorocarbon nanodroplets were investigated.

For this purpose, two different PFC-NDs compositions were characterized using a Malvern Zetasizer Nano-ZS instrument (Malvern Instruments Ltd., UK), measuring sizes (Z-average) and polydispersity (PDI) over time at different stages:
after the dilution (water, 5-folds) performed immediately after their collection from the microfluidic cartridge;
after a storage of one week at 4° C., and
after a storage of one week at −20° C.
   Each measurement was performed at room temperature (i.e. 25° C.).

Two different compositions were tested:
Composition 1: nanodroplets suspension stabilized by the F-TAC surfactant F₈TAC₁₉ and perfluoropentane as PFC; and
Composition 2: nanodroplets suspension stabilized by the Dendri-TAC surfactant DiF₆DiTAC₇ and perfluoropentane as PFC.

The two compositions were prepared as described in Example 1, and the processing parameters were set as to obtain different molar ratios (from low to high) between BFS and PFC for each composition, as illustrated in Table 2. Three different FRR were tested: 3:1, 2:1 and 1:1.

BFS/PFC molar ratios and respective FRR are displayed in the first two columns of Table 2.

Results

TABLE 2
Effects of the BFS/PFC molar ratio on the sizes and PDI of Composition
1 and Composition 2 after 1 week storage at 4° C. or −20° C.
Dilution with water (5-fold)

| Parameters | | Composition 1 | | | | Composition 2 | | | |
|---|---|---|---|---|---|---|---|---|---|
| | Molar | Initial | | | | Initial | | | |
| | ratio | size (nm) | PDI | % Evol | | size (nm) | PDI | % Evol | |
| FRR | nr | 25° C. | | 4° C. | −20° C. | 25° C. | | 4° C. | −20° C. |
| 3-1 | 0.03 | 206.6 | 0.066 | 82 | 62 | 231.7 | 0.115 | 46 | 27 |
| | 0.146 | 208.9 | 0.08 | 37 | 40 | 223.0 | 0.107 | 28 | 17 |
| 2-1 | 0.02 | 222.4 | 0.061 | 102 | 70 | 224.9 | 0.112 | 65 | 42 |
| | 0.097 | 221.7 | 0.064 | 49 | 43 | 213.3 | 0.092 | 43 | 33 |
| 1-1 | 0.04 | 317.4 | 0.034 | 29 | 34 | 301.8 | 0.090 | 21 | 8 |
| | 0.196 | 273.9 | 0.115 | −10 | −1 | 299.3 | 0.088 | −5 | −9 |

The overall results of the study of the effects of the BFS/PFC molar ratios on the calibrated PFC-NDs are displayed in Table 2, which shows a comparison between the sizes and PDI characterization of the two different calibrated NDs compositions obtained after the dilution (water; 5-folds) performed immediately after their collection from the microfluidic cartridge and after a one-week storage at 4° C. or at −20° C.

In particular, the variation of the NDs sizes over time is expressed as % Evol, calculated following Equation 2, as described in the Description Section.

A value of % Evol close to 0 indicates a higher stability of the calibrated NDs suspension, whereby the nanodroplets in the suspension substantially maintain over time its initial mean dimensions.

Results showed that using higher BFS/PFC molar ratios at each predetermined FRR endowed to an improved NDs stability (i.e. low % Evol) over time.

In particular, it was observed that the % Evol parameter has a significant dependence from the molar ratio between BFS and PFC. This effect was particularly evident at the highest molar ratio values (i.e. 0.097, 0.146 and 0.196).

### Example 3

Effects of Dilution Step

The effects of diluting the collected suspension of calibrated perfluorocarbon nanodroplets after their production were investigated.

For this purpose, immediately after their collection from the microfluidic cartridge outlet, the calibrated PFC-NDs suspensions were diluted using water at different dilution coefficients.

PFC-NDs suspensions were characterized using a Malvern Zetasizer Nano-ZS instrument (Malvern Instruments Ltd., UK), measuring sizes (Z average) and polydispersity (PDI) over time at different stages, namely:
immediately after the collecting step of the calibrated PFC-NDs suspensions from the microfluidic cartridge outlet;
after a 2-fold dilution of the calibrated PFC-NDs suspensions with water, and
after a 5-fold dilution of the calibrated PFC-NDs suspensions with water.

Each measurement was performed at room temperature (i.e. 25° C.).

Two different compositions were tested:
Composition 1: nanodroplets suspension stabilized by the F-TAC surfactant F₈TAC₁₉ and perfluoropentane as PFC, and
Composition 3: nanodroplets suspension stabilized by the Dendri-TAC surfactant F₈DiTAC₆ and perfluoropentane as PFC.

Both compositions were prepared as described in Example 1, and the processing parameters were set as to obtain different molar ratios (from low to high) between BFS and PFC for each composition. Three different FRR were tested: 3:1, 2:1 and 1:1.

BFS/PFC molar ratios and respective FRR are displayed in the first two columns of Tables 3 and 4.

Results

The overall results of the calibrated PFC-NDs characterization are displayed in the following Tables 3 and 4.

TABLE 3
Effects of the dilution factor on the initial
sizes and PDI of the Composition 1

| | | | | Dilution with | | Dilution with | |
|---|---|---|---|---|---|---|---|
| Parameters | | No dilution | | water (2-fold) | | water (5-fold) | |
| | Molar | Initial | | Initial | | Initial | |
| | ratio | size (nm) | PDI | size (nm) | PDI | size (nm) | PDI |
| FRR | nr | 25° C. | | 25° C. | | 25° C. | |
| 3-1 | 0.146 | 442.3 | 0.097 | 278.7 | 0.107 | 210.5 | 0.097 |
| 2-1 | 0.097 | 665.2 | 0.099 | 318.7 | 0.065 | 221.1 | 0.090 |
| 1-1 | 0.06 | 1262 | 0.099 | 441.4 | 0.029 | 275.2 | 0.038 |
| | 0.196 | 1574 | 0.074 | 443.7 | 0.072 | 273.9 | 0.115 |

TABLE 4
Effects of the dilution factor on the initial
sizes and PDI of the Composition 3

| | | | | Dilution with | | Dilution with | |
|---|---|---|---|---|---|---|---|
| Parameters | | No dilution | | water (2-fold) | | water (5-fold) | |
| | Molar | Initial | | Initial | | Initial | |
| | ratio | size (nm) | PDI | size (nm) | PDI | size (nm) | PDI |
| FRR | nr | 25° C. | | 25° C. | | 25° C. | |
| 3-1 | 0.146 | 537.2 | 0.110 | 328.2 | 0.100 | 256.7 | 0.104 |
| 2-1 | 0.097 | 832.6 | 0.058 | 361.6 | 0.037 | 264.2 | 0.059 |
| 1-1 | 0.06 | 1428 | 0.127 | 439.0 | 0.046 | 285.8 | 0.090 |
| | 0.196 | 1585 | 0.048 | 471.7 | 0.048 | 323.8 | 0.114 |

Tables 3 and 4 show the sizes and PDI characterization of two different calibrated PFC nanodroplets compositions immediately after the collecting step of the calibrated PFC-NDs suspensions from the microfluidic cartridge outlet and after the dilution performed immediately after their collection.

In particular, the comparison between the sizes and PDI values obtained without dilution and those obtained diluting the samples at two different dilution rates is reported.

Results demonstrate that the dilution step allows reducing the NDs initial sizes (i.e. NDs sizes of the calibrated NDs composition collected from the exit channel of the microfluidic cartridge) for both investigated compositions at each BFS/PFC molar ratio.

Moreover, increasing the dilution coefficient from 2 to 5-fold allowed further reducing the NDs initial size for both investigated compositions at each BFS/PFC molar ratio.

For instance, by performing a 5-fold dilution with water on the Composition 3, it was possible to obtain a reduction of the sizes of about 2 times for the sample with a BFS/PFC molar ratio of 0.146, and of about 5 times for the highest BFS/PFC molar ratio, i.e. 0.196.

### Example 4

Effects of the Dilution with a 5% Trehalose Solution

The effects of diluting the collected suspension of calibrated NDs with an aqueous solution comprising trehalose before the optional freezing step were studied.

For this purpose, immediately after their collection from the microfluidic cartridge, the calibrated PFC-NDs suspensions were diluted using a trehalose aqueous solution at 5% w/w. The investigated dilution coefficient was 5.

After the dilution step, the calibrated NDs were frozen at −20° C. and stored for one week.

PFC-NDs suspensions were characterized using a Malvern Zetasizer Nano-ZS instrument (Malvern Instruments Ltd.UK), measuring sizes (Z average) and polydispersity (PDI) over time at different stages, namely:
after a 5-fold dilution of the calibrated PFC-NDs suspensions using a 5% w/w trehalose aqueous solution, and
after a storage period of one week at −20° C.
   Each measurement was performed at room temperature (i.e. 25° C.).

For this purpose, Composition 1 (F₈TAC₁₉/perfluoropentane) and Composition 2 (DiF₆DiTAC₇/perfluoropentane) were tested.

Both compositions were prepared as described in Example 1, and the processing parameters were set as to obtain different molar ratios (from low to high) between BFS and PFC for each composition. Three different FRR were tested: 3:1. 2:1 and 1:1.

BFS/PFC molar ratios and respective FRR are displayed in the first two columns of Table 5.

Results

The overall results of effects of diluting with an aqueous solution comprising trehalose before the optional freezing step are displayed in Table 5.

TABLE 5
Effects of diluting with Trehalose solution (5% w/w)

| | | Composition 1 | | Composition 2 | |
|---|---|---|---|---|---|
| | | Initial | | Initial | |
| | Molar ratio | size (nm) | % Evol | size (nm) | % Evol |
| FRR | (nr) | 25° C. | −20° C. | 25° C. | −20° C. |
| 3-1 | 0.146 | 237.5 | 15 | 246.7 | 16 |
| 2-1 | 0.097 | 251.6 | 19 | 249.6 | 20 |
| 1-1 | 0.196 | 369.6 | −2 | 350.5 | −8 |

Results demonstrated that the initial NDs sizes were substantially unmodified after one-week storage at −20° C. when diluting the NDs suspensions with a trehalose aqueous solution at 5%.

### Example 5

Influence of the Perfluorocarbon Nature on NDs Sizes and Size Distribution

In order to study the influence of perfluorocarbons on the NDs sizes and size distribution, different compositions comprising different PFC were tested, namely:
Composition 4: comprising a nanodroplets suspension stabilized by the Dendri-TAC surfactant DiF₆DiTAC₇ and perfluorohexane as PFC, and
Composition 5: comprising a nanodroplets suspension stabilized by the Dendri-TAC surfactant F₈DiTAC₆ and perfluorooctylbromide (PFOB).

Composition 4 was prepared as described in Example 1, and the processing parameters were set as to obtain different molar ratios (from low to high) between BFS and PFC for each composition. Three different FRR were tested: 3:1. 2:1 and 1:1. The dilution (water, 5-folds) was performed immediately after their collection from the microfluidic cartridge. The ranges of the evaluated BFS/PFC molar ratios as function of the selected FRR are displayed in the first two left columns of Table 6.

Composition 5 was prepared as described in Example 1, and the processing parameters were set as to obtain a molar ratio of 0.1 between the BFS and PFC: namely, the FRR was 1:1 and the TFR was 15 ml/min. Two PFOB concentrations in the organic phase were tested: 2.5 μL/mL and 10 μL/mL.

Moreover, the stability of Composition 5 was investigated measuring sizes (Z-average) and polydispersity (PDI) at different stages:
after the dilution (water, 4-folds) performed immediately after their collection from the microfluidic cartridge and
after a storage of one week at 4° C.

Each measurement was performed at room temperature (i.e. 25° C.).

The obtained PFC-NDs suspensions (both Composition 4 and Composition 5) were characterized using a Malvern Zetasizer Nano-ZS instrument (Malvern Instruments Ltd., UK), measuring sizes (z-average) and polydispersity (PDI) after the dilution (4-folds) of the collected NDs suspension from the microfluidic outlet.

Results

Table 6 reports the results obtained from the characterization of the microfluidically-prepared Composition 4.

It was observed that using perfluorohexane as PFC in the NDs inner core allowed obtaining monodisperse NDs suspensions, characterized by homogenous nanodroplets in a range size of about 200-250 nm.

TABLE 6
Nanodroplets prepared with composition 4

| | | |
|---|---|---|
| | Initial | |
| Molar ratio | size (nm) | PDI |

| | | |
|---|---|---|
| FRR | (nr) | 25° C. |

| | | | |
|---|---|---|---|
| 3-1 | 0.146 | 192.9 | 0.126 |
| 2-1 | 0.097 | 199.2 | 0.109 |
| 1-1 | 0.196 | 234.5 | 0.122 |

Table 7 reports the results obtained from the characterization of the microfluidically-prepared Composition 5.

TABLE 7
Nanodroplets prepared with Composition 5

| | | | |
|---|---|---|---|
| PFC conc. in | Initial | | |
| the org. phase | size (nm) | PDI | % Evol |

| | | | |
|---|---|---|---|
| Molar ratio | (μL/mL) | 25° C. | 4° C. |

| | | | | |
|---|---|---|---|---|
| 0.1 | 2.5 | 273 | 0.058 | 12 |
| | 10 | 280 | 0.089 | 14 |

The characterization of Composition 5 further demonstrated that using perfluoroctylbromide as PFC in the NDs inner core led to good results in term of NDs size. Moreover, the determined PDI values confirmed the monodispersity of the NDs suspensions.

Results showed that using perfluoroctylbromide (PFOB) as PFC endowed to a good NDs stability (i.e. low % Evol) over time.

### Example 6

Repeatability of the Microfluidic Method

In order to assess the repeatability of the disclosed microfluidic method, multiple formulations of calibrated perfluorocarbon nanodroplets were prepared as described in Example 1 and subsequently characterized.

The term repeatability indicates a measure of the ability of the microfluidic method to generate similar results for multiple preparations of the same sample.

For this purpose, five samples of PFC-NDs were prepared having the same composition, i.e. Composition 2 (DiF₆DiTAC₇/perfluoropentane) with a molar ratio of 0.196; the set processing parameters were FRR 1-1 and TFR 15 mL/min.

Immediately after their collection from the microfluidic cartridge outlet, the samples were diluted 5-folds using water and were characterized using a Malvern Zetasizer Nano-ZS instruments in order to determine the NDs sizes (Z-Average) and polydispersity (PDI).

Results

TABLE 8
Characterization of microfluidically-prepared
multiple formulations of Composition 2

| | | |
|---|---|---|
| Molar ratio | Initial size (nm) | PDI |

| | | |
|---|---|---|
| FRR | n | 25° C. |

| | | | |
|---|---|---|---|
| 1-1 | 0.196 | 239.4 | 0.110 |
| | | 229.6 | 0.098 |
| | | 213.8 | 0.079 |
| | | 235.1 | 0.083 |
| | | 236.3 | 0.084 |

| | | |
|---|---|---|
| Average | 230.8 ± 10.2 | 0.091 ± 0.013 |

The characterization of the microfluidically-prepared multiple formulations having the same composition (Table 8) demonstrated that the repeated samples including PFC-NDs had similar values of sizes and PDI. The repeatability coefficient, that is to say the maximum difference that is likely to occur between repeated measurements, was calculated as coefficient of variation to be below 5%. The coefficient of variation, expressed as a percentage, is the ratio of the standard deviation and the overall mean of the repeated measurements.

These results confirmed the high repeatability of the disclosed microfluidic method.

### Example 7

Influence of a BFS Mixture on NDs Sizes and Size Distribution

In order to study the influence of a BFS mixture as stabilizing agent on the NDs sizes and size distribution, a mixture of two different BFS, namely an amphiphilic linear oligomer FTAC and a dendrimer DendriTAC, was added to the aqueous phase.

For this purpose, the following composition was prepared:
Composition 6: nanodroplets suspension stabilized by a mixture of a F-TAC surfactant, i.e. F₈TAC₁₉ and a Dendri-TAC surfactant, i.e. DiF₆DiTAC₇; perfluoropentane was used as PFC. The molar ratio between BFS and PFC was 0.1, and the set processing parameters were a FRR of 1-1 and a TFR of 15 mL/min.

Different molar ratios (%) between F₈TAC₁₉ and DiF₆DiTAC₇ were tested: 75:25, 50:50 and 25:75.

Immediately after the collection from the microfluidic cartridge outlet, the calibrated PFC-NDs suspensions were diluted 5-folds using water and were characterized using a Malvern Zetasizer Nano-ZS instrument in order to determine the NDs sizes (Z-Average) and polydispersity (PDI).

Results

TABLE 9
Influence of the BFS mixture (F₈TAC₁₉ and
DiF₆DiTAC₇) on NDs sizes and size distribution.
Composition 6

| | |
|---|---|
| Initial size | |
| (nm) | PDI |

| | | |
|---|---|---|
| F₈TAC₁₉ | DiF₆DiTAC₇ | 25° C. |

| | | | |
|---|---|---|---|
| 100% | 0% | 285 | 0.037 |
| 75% | 25% | 279 | 0.076 |
| 50% | 50% | 248 | 0.075 |
| 25% | 75% | 257 | 0.122 |
| 0% | 100% | 236 | 0.095 |

The results showed that using a BFS mixture as stabilizing agent in the microfluidically-prepared PFC-NDs allowed to obtaining good PDI values, thus confirming the good monodispersity of the nanodroplets in suspension.

No relevant differences in terms of NDs sizes and PDI were observed using different molar ratios between F₈TAC₁₉ and DiF₆DiTAC₇.

### Example 8

Acoustic Droplet Vaporization Determination

The Acoustic Droplet Vaporization (ADV) threshold of the NDs prepared according to the previous examples can be determined according to conventional methodologies using B-mode imaging methods.

For instance, the suspension of NDs can be vaporized while passing through the focal zone of a transducer and the acoustic pressure is increased of about 0.2 MPa each 10 s (starting at about 3.0 MPa) until the NDs vaporization is observed. The NDs suspensions prepared according to the previous examples show a value of acoustic vaporization of from about 4.2 to 4.6 MPa using a single-element transducer at 6.0 MHz. Pulses were emitted in burst mode, 200 cycles per pulse, at a pulse-repetition frequency (PRF) of 10 Hz.

### REFERENCES

Astafyeva et al, J. Mater. Chem. B, 3, 2015, 2892-2907 WO2016185425
Sheeran et al., IEEE T ULTRASON FERR, 64, 1, 2017, 252-263
Melich et al., International Journal of Pharmaceutics, 587, 2020, 119651
Helfield et al., Ultrasound in Medicine & Biology, 46, 10, 2020, 2861-2870

1) A nanodroplet comprising an outer layer and an inner core, said outer layer comprising a biocompatible fluorinated surfactant and said inner core comprising a fluorocarbon, characterized in that the molar ratio between said biocompatible surfactant and said fluorocarbon is higher than 0.06, wherein said biocompatible fluorinated surfactant is selected from
(A) an amphiphilic dendrimer (Dendri-TAC) of generation n comprising:
a hydrophobic central core of valence 2 or 3;
generation chains [symbol] attached to each respective open end of the central core and branching around the core; and
a hydrophilic terminal group at the end of each generation chain;
wherein
n is an integer from 0 to 12 and identifies the hydrophilic terminal group comprising:
a mono-, oligo- or polysaccharide residue,
a cyclodextrin residue,
a peptide residue,
a tris(hydroxymethyl)aminomethane (Tris), or
a 2-amino-2-methylpropane-1,3-diol;
the hydrophobic central core is a group of formula (Ia) or (Ib) 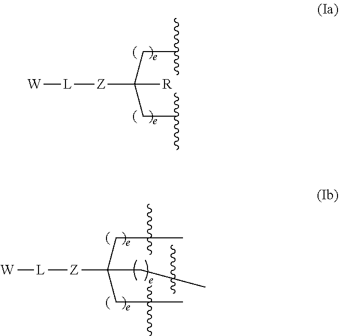
wherein:
W is R_{F} or a group selected from W₀, W₁, W₂ or W₃: 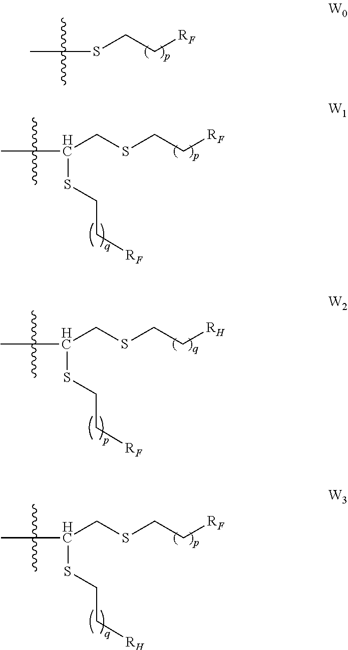
R_{F} is a C₄-C₁₀ perfluoroalkyl or a C₁-C₂₄ alkyl group,
R_{H} is a C₁-C₂₄ alkyl group,
p is 0, 1, 2, 3 or 4;
q is 0, 1, 2, 3 or 4;
L is a linear or branched C₁-C₁₂ alkylene group, optionally interrupted by one or more —O—, —S—,
Z is C(═O)NH or NHC(═O),
R is a C₁-C₆ alkyl group, and
e is at each occurrence independently selected from 0, 1, 2, 3 or 4,
(B) an amphiphilic linear oligomer (F-TAC) of formula II 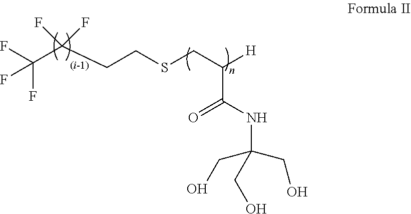
wherein:
n is the number of repeating Tris units (n=DPn is the average degree of polymerization), wherein the term Tris indicates the tris(hydroxymethyl)aminomethane unit, and
i is the number of carbon atoms in the fluoroalkyl chain or a mixture thereof.

2) The nanodroplet according to claim 1, wherein said amphiphilic dendrimer Dendri-TAC is selected from the group comprising the following compounds of formula IA 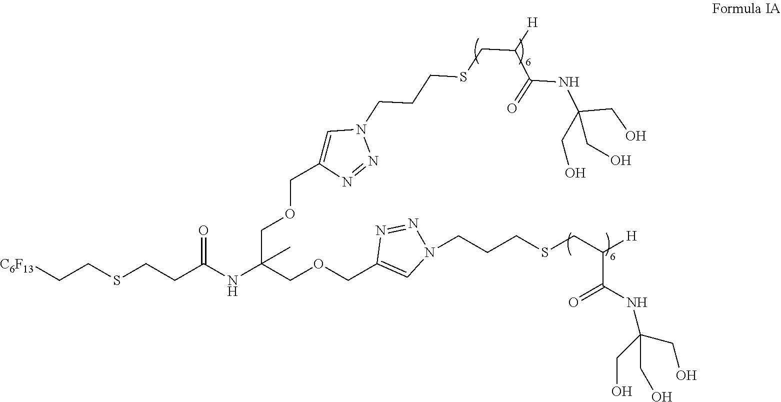
and of formula IB

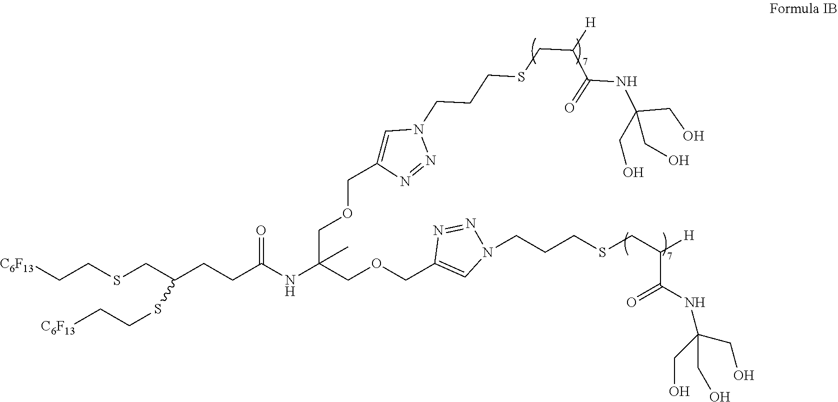

3) The nanodroplet according to claim 1, wherein said amphiphilic linear oligomer F-TAC is selected from the group comprising the following compounds of formula IIA 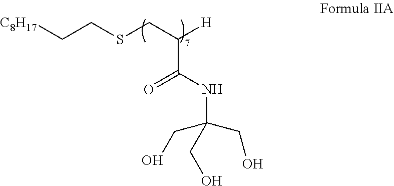
and of formula IIB

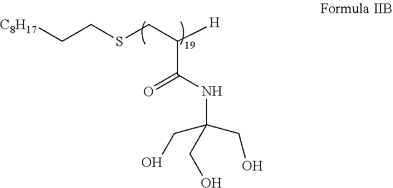

4) The nanodroplet according to claim 1, wherein said fluorocarbon is a perfluorocarbon.

5) The nanodroplet according to claim 1, wherein said molar ratio between said biocompatible fluorinated surfactant and said fluorocarbon is higher than 0.07.

6) An aqueous suspension comprising a nanodroplet according to claim 1.

7) An aqueous suspension comprising a plurality of nanodroplets according to claim 1, wherein said nanodroplets have a z-average diameter comprised between 100 nm and 1000 nm and a polydispersity lower than 0.25.

8) The aqueous suspension according to claim 6 further comprising trehalose.

9) A method for the preparation of an aqueous suspension of calibrated fluorocarbon nanodroplets, said method comprising the steps of:
a) Preparing an aqueous phase;
b) Preparing an organic phase,
wherein
i) said aqueous phase comprises a biocompatible fluorinated surfactant selected from a Dendri-TAC, a F-TAC or a mixture thereof and the organic phase comprises a fluorocarbon or
ii) said organic phase comprises a biocompatible fluorinated surfactant selected from a Dendri-TAC, a F-TAC or a mixture thereof and a fluorocarbon.
c) Injecting said aqueous phase into a first inlet and said organic phase into a second inlet of a microfluidic cartridge, thereby mixing said aqueous phase and said organic phase in a mixing device of the microfluidic cartridge to obtain an aqueous suspension of calibrated fluorocarbon nanodroplets, and
d) Collecting the aqueous suspension of calibrated fluorocarbon nanodroplets from the exit channel of the microfluidic cartridge.

10) The method according to claim 9, wherein said aqueous phase comprises a biocompatible fluorinated surfactant selected from a Dendri-TAC, a F-TAC or a mixture thereof and said organic phase comprises a fluorocarbon.

11) The method according to claim 9, wherein said fluorocarbon is a perfluorocarbon.

12) The method according to claim 11, wherein said perfluorocarbon is selected from perfluoropentane, perfluorohexane, perfluorooctylbromide or a mixture thereof.

13) The method according to claim 9, wherein the ratio between the volume of said aqueous phase and the volume of said organic phase is comprised between 1:1 to 5:1.

14) The method according to claim 9, further comprising additional step e) wherein said collected suspension of calibrated fluorocarbon nanodroplets is diluted with an aqueous liquid.

15) The method according to claim 14, wherein said aqueous liquid is water.

16) (canceled)

17) (canceled)

18) (canceled)

19) An aqueous suspension comprising a plurality of calibrated fluorocarbon nanodroplets obtainable by a method of preparation comprising the steps of:
a) Preparing an aqueous phase;
b) Preparing an organic phase,
wherein i) said aqueous phase comprises a biocompatible fluorinated surfactant, selected from Dendri-TAC, F-TAC or a mixture thereof, and the organic phase comprises a fluorocarbon or
ii) said organic phase comprises a biocompatible fluorinated surfactant, selected from selected from Dendri-TAC, F-TAC or a mixture thereof and a fluorocarbon.
c) Injecting said aqueous phase into a first inlet and said organic phase into a second inlet of a microfluidic cartridge, thereby mixing said aqueous phase and said organic phase in a mixing device of the microfluidic cartridge to obtain an aqueous suspension of calibrated fluorocarbon nanodroplets, and
d) Collecting the aqueous suspension of calibrated fluorocarbon nanodroplets from the exit channel of the microfluidic cartridge,
wherein said nanodroplets have a z-average diameter comprised between 100 nm and 1000 nm and a polydispersity lower than 0.25.

20) (canceled)

21) A method of diagnostic and/or therapeutic treatment comprising administering the aqueous suspension according to claim 6 to a patient.
